(19) United States
(12) Patent Application Publication
(10) Pub. No.: US 2016/0052993 A1
(43) Pub. Date: Feb. 25, 2016

(54) ALBUMIN VARIANTS BINDING TO FCRN

(71) Applicant: ELEVEN BIOTHERAPEUTICS, INC., Cambridge, MA (US)

(72) Inventors: Michael March Schmidt, Boston, MA (US); Sharon Townson, Cambridge, MA (US); Thomas M. Barnes, Brookline, MA (US)

(21) Appl. No.: 14/888,667

(22) PCT Filed: May 2, 2014
(86) PCT No.: PCT/US2014/036508

§ 371 Date: Nov. 2, 2015

Related U.S. Application Data
(60) Provisional application No. 61/826,726, filed on May 23, 2013.
(60) Provisional application No. 61/819,099, filed on May 3, 2013.

(51) Int. Cl. C07K 14/765; G01N 33/68; C07K 16/18

(52) U.S. Cl. CPC C07K 14/765; C07K 16/18; G01N 33/68; G01N 33/6872; G01N 2333/765; G01N 2333/70535

(57) ABSTRACT

The invention relates to methods of identifying albumin variants having improved pharmacokinetics, albumin variants having improved pharmacokinetics, and therapeutic uses of albumin variants having improved pharmacokinetics.

### RELATED APPLICATIONS

This application claims priority to U.S. Application Ser. No. 61/819,099, filed May 3, 2013 and to U.S. Application Ser. No. 61/826,726, filed May 23, 2013. The entire contents of each of the foregoing applications are hereby incorporated herein by reference.

### FIELD OF THE INVENTION

The field of the invention relates to protein metabolism. More particularly, the field relates to albumin and FcRn recycling

### BACKGROUND

Serum albumin (SA) is the most abundant protein in mammalian plasma, and binds many endogenous and exogenous molecules such as fatty acids and lipophilic small molecules, at over ten discrete sites. SA and immunoglobulin G (IgG) have circulating half-lives that are longer than those of other circulating proteins; about 18 and 22 days in humans for SA and IgG, respectively, compared with, for example, about 3-6 days for other Ig classes. This arises from a shared property. SA and IgG can be rescued from degradation by the neonatal Fc receptor (FcRn). In general, fluid phase endocytosis in endothelial and myeloid cells continuously removes plasma proteins to an acidic endosomal compartment, whence they are sorted to the lysosome and degraded. In the case of SA and IgGs, a pH-dependent interaction occurs between these proteins and FcRn, a transmembrane protein that sorts to the cell surface and does not enter the lysosomal degradation pathway. At the cell surface, the bound proteins are released upon titration back to physiological pH, at which they rapidly dissociate from FcRn. Based on modeling, it has been estimated that FcRn recycles six HSA molecules for every IgG, and the ratio is 30:1 in mice.

The pH-dependent interaction between FcRn and IgG1 has been studied in some detail. FcRn is a heterodimer of a non-polymorphic MHC class I-like α chain and β2 microglobulin (β2m; FIG. 5A). No significant conformational change occurs upon pH shift in either IgG1 or the FcRn interface; rather, the interaction is mediated by protonation of key histidine residues in the C_{H}2-C_{H}3 hinge region of IgG1, which then form salt bridges with key acidic residues at the FcRn interface. FcRn can bind IgG and SA simultaneously, with neither competition nor cooperation, indicating a distinct, independent pH-dependent binding site. Consistent with this, the SA-FcRn interaction is detergent-sensitive and hydrophobic in character, while the IgG-FcRn interaction is detergent insensitive and largely polar.

### SUMMARY

The invention relates to the discovery of the detailed structural relationship between albumin and FcRn, methods of improving albumin pharmacokinetics (PK) by increasing affinity for FcRn at endosomal pH, decreasing fatty acid binding to albumin, and albumin variants having such improved PK (e.g., increased PK, as indicated by, e.g., improvements in one or more pharmacokinetic parameters, e.g., as indicated by increased half-life or decreased clearance). Accordingly, the invention relates to a method of identifying a human serum albumin (HSA) variant. The method includes, providing a mutated HSA; and determining whether the mutated HSA has at least one mutation in domain III that decreases fatty acid binding compared to fatty acid binding by a wild type HSA, wherein a mutated HSA that decreases fatty acid binding compared to a wild type HSA is an HSA variant. Further, the method can include determining the binding affinity of the mutated HSA for FcRn, wherein a mutated HSA that can bind to FcRn with the same or increased affinity compared to binding of a wild type HSA to FcRn is an HSA variant. The method can also include determining the PK of the mutated HSA compared to the PK of a wild type HSA, wherein a mutated HSA that has increased PK compared to a wild type HSA is an HSA variant.

In some embodiments, the invention relates to a human serum albumin (HSA) variant that includes at least one mutation in domain III that decreases fatty acid binding to the HSA variant compared to fatty acid binding by a wild type HSA. In some cases, the HSA variant can bind to FcRn. In some embodiments, the HSA variant has an increased PK compared to a wild type HSA. In some cases, the mutation alters one or more residues in domain III of a wild type HSA that can bind to a carboxyl; or alters one or more residues in domain III that are lining residues. The HSA variant is, in some cases, mutated at one or more residues selected from the group consisting of R410, Y411, S489, Y401, and K525. The mutation can be to a non-polar amino acid or a negatively charged amino acid, e.g., alanine or glutamic acid, respectively.

In some embodiments, the HSA variant has mutation in one or more lining residues selected from the group consisting of Y411, V415, V418, T422, L423, V426, L430, L453, L457, L460, V473, R485, F488, L491, F502, F507, F509, K525, A528, L529, L532, V547, M548, F551, L575, V576, S579, and L583. In some cases, the mutated residue is selected from the group consisting of Y411, V415, V418, L423, V426, L430, L453, L457, L460, V473, P485, F488, L491, F502, F507, F509, A528, L529, L532, V547, M548, F551, L575, V576, and L583. The mutated residue is, in some embodiments, mutated to a serine.

An HSA variant can be associated with or attached to a therapeutic agent (e.g., a biologic or small molecule therapeutic). The association or attachment can be any known in the art. For example an HSA variant can be covalently linked to a therapeutic agent (e.g., a protein, peptide or small molecule). In embodiments, an HSA variant is expressed as a heterologous protein. In embodiments, the HSA variant improves the PK of the therapeutic agent.

The invention also relates to a method of identifying a scaffold molecule, the method comprising providing a candidate molecule; and determining whether the candidate molecule can bind to an HSA and can inhibit fatty acid binding to the HSA, wherein, a candidate molecule that can bind to an HSA and can inhibit fatty acid binding is a scaffold molecule. In some cases, the scaffold molecule can bind to one or more of residues R410, Y411, S489, Y401, or K525 of a wild type HSA. The invention also relates to a scaffold molecule, e.g., a molecule identified by a method described herein. In some embodiments, the scaffold molecule further comprises a therapeutic molecule, thereby forming a heterogeneous scaffold molecule, wherein the PK of the heterogeneous scaffold molecule is increased compared to the PK of the therapeutic molecule.

Also provided herein is a method of increasing the serum half-life of a molecule, the method comprising linking, e.g., covalently linking, the molecule to an HSA variant described herein. In embodiments, the molecule is a protein or polypeptide.

Also provided herein is a molecule comprising an HSA variant described and a heterologous molecule. In embodiments, the heterologous molecule is a protein or polypeptide.

The entire disclosure of each patent document and scientific article referred to herein, and those patent documents and scientific articles cited thereby, is expressly incorporated by reference herein for all purposes.

Additional features and advantages of the invention are more particularly described below.

### BRIEF DESCRIPTION OF THE DRAWINGS

FIG. 1A is a diagram of a selection scheme for isolation of high affinity HSA variants.

FIG. 1B is a reproduction of a FACS plot of a selected mutagenized clone pool at pH 5.5 after four rounds of sorting (right panel) compared to the starting library (left panel).

FIG. 1C is a bar graph illustrating the binding-display ratio evolution of clone pools binding to 10 nM schFcRn (left panel) and binding-fluorescence reflecting pH-dependent behavior of clone pools.

FIG. 2A is a drawing of a representation of the WW loop of the HSA13/hFcRn complex.

FIG. 2B is a drawing of a representation of the DI/hFcRnα contact of the HSA13/hFcRn complex.

FIG. 3A is a drawing of a representation of the hydrophobic contact, the W59 pocket. HSA13 mutations are italicized. C12:0 (sphere furthest to the left), C16:0 (sphere in the middle) and C18:1 (sphere furthest to the right) fatty acids (from PDB codes 1BJ5, 1E7H and 1GNI) are shown, with the van der Waal's radius displayed for the terminal atom. The position of W59 from unbound hFcRn (structure at right that includes W59; from PDB code 3M17) is also shown.

FIG. 3B is a graph of SPR traces showing how hydrophobic contact mutations W59A and W59F in FcRn affect HSA binding (HSA immobilized) and a table of ELISA data (K_{D} values, in nM) for HSA13 (HSA13 immobilized) binding similarly.

FIG. 3C is a graph of results of HSA bearing fatty acids, C12:0, C16:0 and C18:1, binding to immobilized hFcRn. C16:0 and C18:1 bind poorly.

FIG. 3D is a drawing illustrating the W53 pocket of HSA. Two thyroxines are drawn in sites 2 and 3 (from PDB code 1HK3).

FIG. 3E is a graph depicting the results of an experiment testing the binding of HSA and hFcRn mutations W53A and W53F mutations and wild type hFcRn (SPR traces; HSA immobilized) and a table inset showing ELISA data (K_{D} values, in nM) with HSA13 (HSA13 immobilized) binding similarly. The effects of T4 binding could not be evaluated due to its low solubility and low HSA affinity.

FIG. 4A to E show pH-dependent binding. FIG. 4A: ^{hFcRnα}H166 environs are presented. The E165/R169 hydrogen bond shown here is not present in uncomplexed hFcRn. FIG. 4B: The HH loop in HSA13. apo HSA (PDB code 1AO6) is light gray. FIG. 4C: ^{HSA}H510 environs showing protonation-dependent bonds. His-510 forms a p-cation bond with ^{hFcRnα}Trp-176 (upper gray dotted line). FIG. 4D: ^{HSA}H535 environs showing protonation-dependent bonds. FIG. 4E: Model for pH-dependent association. At pH 7.4, the WW loop is disordered and the HH loop is loosely structured. Upon shift to pH 6, histidines become protonated and make hydrogen bonds (black circles and lines), stabilizing the two surfaces. The DIA and W59 contacts drive the initial interaction, which then engages DIIIB to pull open the W53 pocket.

FIG. 5A is a drawing of two views of human FcRn with the alpha chain in light gray and the beta chain (which is β2m) in darker gray. The end-on view illustrates the narrowing of the helices in the MHC class I fold due to a warp in the α1α2 platform.

FIG. 5B is a drawing of defatted HSA PDB code 1AO6 shown in the classical “heart” orientation. DIA DIB, DII, DIIIA, DIII loop, and DIIIB are labeled and shown in different shades of gray.

FIG. 6A depicts alignments of proteins from 9 mammals: human, macaque, cow, mouse, rat, rabbit, horse, dog, and pig. a) Portions of SA, covering the contacts in DI and DIII. Contacts to FcRnα are in the fine boxes, contacts to β2m are in boldface boxes, and residues that contact both are in a black background with white lettering. The HH loop is shown. The four positions that are changed in HSA13 are underlined. The histidines at positions 440, 464, 510 and 535 (human numbering) are bold.

FIG. 6B depicts portions of FcRnα, covering the contacts to DI and DIII. Contacts to HSA DI are in the fine boxes, contacts to DIII are in boldface boxes, and residues that contact both are shown in a black background with white lettering. The WW loop is shown. The histidines at positions 161 and 166 (human numbering) are bold. Half (6/12) of all human/mouse interfacial sequence differences cluster in the region of FcRn that contacts DI (N/R149, L/S152, T/E153, F/T157, H/E161, E/G165, human numbering), showing that this contact is overall not highly conserved. Nonetheless, since hFcRn has higher affinity than mouse FcRn for either HSA or mouse SA33, the systematic superiority of hFcRn could be due to some of these changes.

FIG. 6C depicts the complete β2m. Contacts to HSA DIII are shown in a black background with white lettering. For all alignments, if the residue in a non-human species is identical to human, it follows the same tonal/formatting scheme.

FIG. 7A is a drawing depicting ^{HSA}K573 environs. K573 makes a salt bridge to ^{β2m}E69, stabilized by contacts to ^{β2m}S20 and the ^{β2m}N21 backbone carbonyl.

FIG. 7B is a drawing depicting ^{HSA}G505 environs. G505 makes a contact to ^{hFcRnα}S230, but the DIII loop in this area is likely not in its natural FcRn-bound position. Apo HSA (1AO6, white) shows where the wild-type residue, E505 would sit, and shows that E505R, which also improves affinity similarly to E505G (Table 2, compare HSA6 to HSA16), could make a salt bridge to ^{hFcRnα}D231.

FIG. 8A is a graph depicting binding data for histidine mutants of hFcRn or HSA at pH 6.0 Mutations at H166 of hFcRnα significantly reduce binding to both wild type HSA and HSA13 as measured by SPR (HSA immobilized) and ELISA (HSA13 immobilized, inlaid table), respectively.

FIG. 8B is a graph depicting binding data for mutations of ^{HSA}H510 and ^{HSA}H535 to Phe which reduced hFcRn binding similarly, whether in wild type HSA (SPR) or HSA13 (ELISA); schFcRn immobilized for both.

FIG. 8C is table of data generated for mutations at ^{hFcRnα}H161 and ^{HSA}H464, demonstrating these mutations have more minor effects; HSAs were immobilized.

### DETAILED DESCRIPTION

The long circulating half-life of serum albumin, the most abundant protein in mammalian plasma, derives from pH-dependent endosomal salvage from degradation, mediated by the neonatal Fc receptor, FcRn. This property has been exploited to extend the half-lives of rapidly cleared therapeutic proteins by fusion to human serum albumin (HSA). However, it is useful to identify additional albumins having improved affinities for FcRn and able to preserve desirable properties such as increased affinity when fused to another entity such as a therapeutic agent.

Applicants have solved the co-crystal structure of human FcRn (hFcRn) bound to a high-affinity variant of HSA, at pH 4.9 to 2.4 Å resolution. Previously, the crystal structure of the HSA-FcRn had not been solved.

The high affinity HSA variant used in solving the structure described herein was one of several developed by applicants that showed up to a 300-fold increase in hFcRn affinity at pH 6. Applicants therefore also evaluated whether high-affinity HSA variants also had increased circulating half-lives.

The HSA-FcRn complex structure was discovered to have an extensive, primarily hydrophobic interface featuring two key FcRn tryptophan side chains inserting into deep hydrophobic pockets on HSA, and stabilized by hydrogen-bonding networks involving protonated histidines internal to each protein. Each pocket is near or overlaps with albumin ligand binding sites. It was also discovered that fatty acid ligands can compete with FcRn, suggesting that some liganded albumin species do not recycle. Furthermore, the high affinity HSA variants demonstrate significantly increased circulating half-lives in mice and monkeys. These findings clarify fundamental aspects of albumin biology and provide methods for creating biotherapeutics with improved pharmacokinetics.

The overall architecture of the low-pH HSA/hFcRn complex that was discovered by applicants and is described herein accounts for the high entropic gain upon binding, due to its dependence on hydrophobic interactions (Chaudhury et al. (2006) Biochem 45:4983-4990).

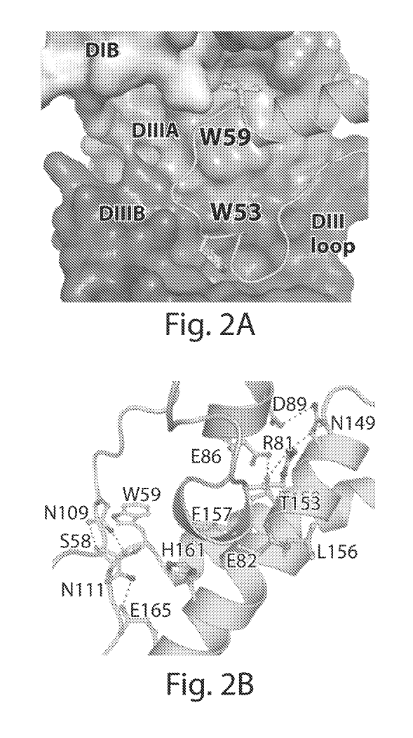

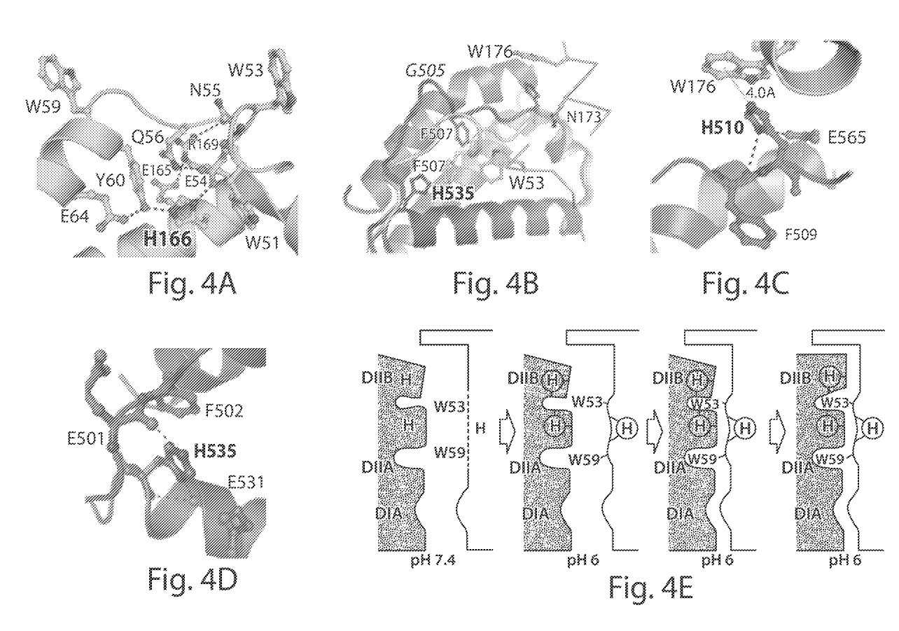

There were several surprising features of the interaction. First was the extensiveness of the interaction (e.g., see FIG. 2A, and FIG. 2B), given the low affinity of wild type HSA, and the absence of any meaningful direct pH-dependent contacts (FIG. 4A to E), compared, for example, to the rat IgG1-hFcRn interaction, which features four titratable salt bridges and is half the size (Martin et al. (2001) Mol Cell 7:867-877). Instead, a fundamentally ionic shift results in the elaboration of a hydrophobic surface that provides most of the energy, with a prominent use of aromatic side chains to make profound hydrophobic contacts or engage in π interactions.

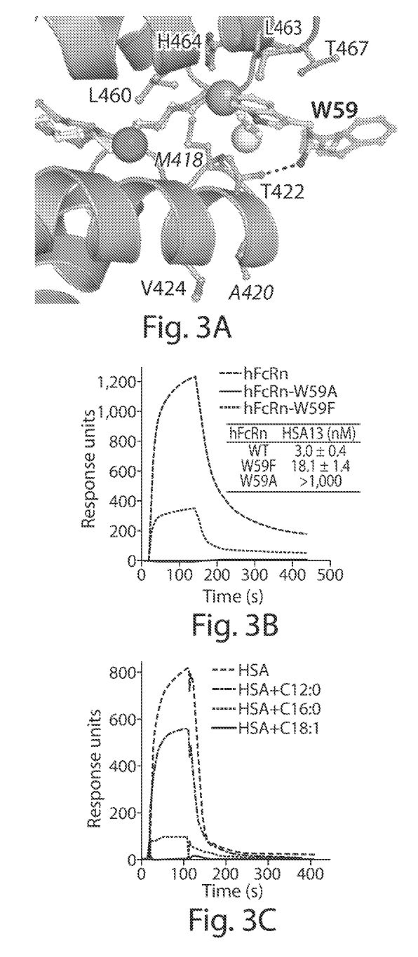

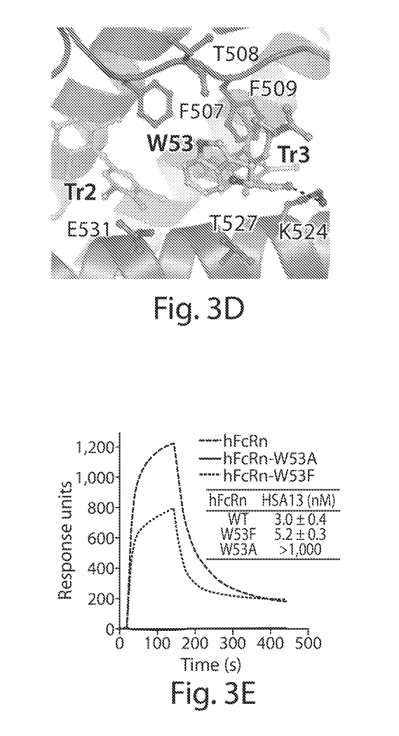

Second was the site overlap and competition between natural ligands and hFcRn, implicating bound ligands as direct controllers of the circulating half-life of HSA via molecular mimicry (FIG. 3A to E). Upon ligand binding, structural changes can propagate through HSA to affect affinities at other sites, indicating that ligands bound to Drug Site 2, near the W59 pocket, also influence recycling efficiency. Thus, selective non-salvage of certain liganded species provides an unanticipated means to deliver those ligands to the up-taking cells upon degradation of SA. Consequently, mutations that exclude ligands from these sites can be created that enhance recycling, without affecting FcRn affinity.

HSA is reportedly recycled inefficiently, because of hFcRn saturation, and as with IgGs, increasing the affinity of HSA for hFcRn can increase its circulating half-life. In analbuminemic people, non-saturating doses of HSA are reported to exhibit half-lives of 50-100 days, which represents the limit attainable in normal people by increase of the low-pH on-rate. General improvements in affinity run the risk of acquiring neutral pH binding, but HSA appears to be able to uncouple its pH 7.4 and 6.0 hFcRn affinities (Table 1, infra. This indicates that longer half-life gains can be achieved by further increasing the affinity for hFcRn at low pH with the appropriate counterselection at pH 7.4. The large contact surface only provides a 1-5 μM K_{D} at pH 6.0 for wild-type HSA (Chaudhury et al. (2006) Biochem 45:4983-4990; Andersen et al. (2006) Eur J Immunol 36:3044-3051; Table 2 infra), which provides that methods of influencing the affinity of an HSA can be achieved via gain, loss or tuning of contacts.

Accordingly, in some embodiments methods are provided for identifying and/or designing an HSA variant that has an altered half-life, e.g., an increased half-life. In some cases, the increased half-life is achieved by decreasing the ability of the HSA to bind to a ligand that can compete for an FcRn site, e.g., decreasing the ability of a long chain fatty acid to bind to the HSA variant. This approach is in contrast to an approach that increases half-life by manipulating the binding affinity of HSA and FcRn, although in some embodiments, both approaches can be applied to identify an HSA variant with increased half-life.

In general, at least one mutated HSA is provided. Methods for generating such molecules are known in the art. At least one, two, or three of the following features are determined for the HSA(s): whether the mutated HSA has at least one mutation in domain III that decreases fatty acid binding compared to fatty acid binding by a wild type HSA, whether the binding affinity of the mutated HSA for FcRn is the same or increased compared to binding of a wild type HSA to FcRn, whether the mutated HSA has increased PK (e.g., an increased half-life) compared to the PK of a wild type HSA.

Further, in some embodiments, an HSA variant is a mutated HSA that has one or more of the following features: at least one mutation in domain III that decreases fatty acid binding to the HSA variant compared to fatty acid binding by a wild type HSA, the HSA variant can bind to FcRn with at least the same affinity as a wild type HSA, and the HSA variant has an increased PK compared to a wild type HSA. The HSA variant can, in some embodiments, have one or more altered residues in domain III that can bind to a carboxyl, e.g., at R410, Y411, S489, Y401 or K525. The residues are, in some cases, mutated to a non-polar amino acid or a negatively charged amino acid, e.g., an alanine or a glutamic acid, respectively.

In some cases, the HSA variant has an alteration (e.g., a mutation) to one or more residues in domain III that are lining residues. A lining residue in domain III is, for example, Y411, V415, V418, T422, L423, V426, L430, L453, L457, L460, V473, R485, F488, L491, F502, F507, F509, K525, A528, L529, L532, V547, M548, F551, L575, V576, S579, and L583. For example, the mutated lining residue can be Y411, V415, V418, L423, V426, L430, L453, L457, L460, V473, P485, F488, L491, F502, F507, F509, A528, L529, L532, V547, M548, F551, L575, V576, and L583. In some cases the mutated residue is mutated to a serine.

Therapeutic Uses of HSA Variants

An HSA variant can be used as a therapeutic, e.g., in uses for which albumin such as human albumin is typically used. For such uses, an HSA variant as described herein can have the advantage of extended PK, which can enable less frequent and/or reduced dosing for albumin replacement or supplementation. Such uses include, for example, hypovolemia, hypoalbuminemia, burns, adult respiratory distress syndrome, nephrosis, and hemolytic disease of the newborn. Hypoalbuminemia can result from, for example, inadequate production of albumin (e.g., due to malnutrition, burns, major injury, or infection), excessive catabolism of albumin (e.g., due to burn, major injury such as cardio-pulmonary bypass surgery, or pancreatitis), loss through bodily fluids (e.g., hemorrhage, excessive renal excretion, or burn exudates), deleterious distribution of albumin within the body (e.g., after or during surgery or in certain inflammatory conditions). Typically, for such uses, an HSA variant for such uses is administered by injection or iv in a solution that is from 5%-50% HSA variant (w/v), for example, 10%-40%, 15%-30%, 20%-25%, 20%, or 25%. Typically, administration is sufficient to produce a total albumin plus HSA variant concentration in a treated subject's serum that is about 3.4-5.4 grams per deciliter (g/dL). Methods of assaying albumin concentration are well known in the art and can generally be used to assay total albumin plus HSA variant concentration.

Uses of HSA Variants in Association with Other Agents

HSA variants can also be used in association with other agents, e.g., therapeutic or diagnostic agents, to confer functional advantages, e.g., advantages of HSA variants as described herein. The agent can be, e.g., any agent that is useful in the diagnosis or therapy of a disease or disorder, e.g., a disease or disorder that affects a human or a non-human animal.

Advantages of HSA variants include, e.g., lack of Fc effector function, high solubility, potential for high expression, low immunogenicity, and ability to be fused to another moiety at both termini to generate bivalent or bispecific molecules.

Disclosed herein are HSA variants with improved half-life, thereby potentiating the ability of the HSA variant and an agent associated with that HSA variant to have improved pharmacokinetics (PK).

An HSA variant that has an extended PK can be associated with an agent (e.g., a therapeutic or diagnostic agent) to extend the PK of the agent. The extended PK can have advantages; for example, the agent can be administered less frequently and/or at reduced concentrations and/or more consistent delivery levels of the agent can be achieved.

In some embodiments, associating an HSA variant with an agent improves the functional properties of the agent. In some embodiments, the dosage and/or frequency at which the agent is effective for producing a particular effect (e.g., a desired therapeutic effect) is reduced when the agent is used in association with the HSA variant. In some embodiments, associating an HSA variant with an agent improves the pharmacokinetic properties of the agent (e.g., increases its half-life and/or reduces its clearance). Any relevant pharmacokinetic parameters that are known in the art can be used to assess pharmacokinetic properties. The pharmacokinetics of an HSA variant or an HSA variant associated with an agent can be measured in any relevant biological sample, e.g., in blood, plasma, or serum.

In some embodiments, the dose at which the agent is effective for producing a particular effect (e.g., a desired therapeutic effect) is reduced when the agent is associated with the HSA variant. In some embodiments, the effective dose is reduced to 80%, 70%, 60%, 50%, 40%, 30%, 20%, or 10% of the dose that is required when the agent is not associated with the HSA variant.

In some embodiments, the frequency of dosing of the agent that is effective for producing a particular effect (e.g., a desired therapeutic effect) is reduced when the agent is associated with the HSA variant. In some embodiments, the frequency of dosing at which the agent is effective when it is associated with the HSA variant is decreased by 10%, 20%, 30%, 40%, 50%, or more compared with the frequency at which the agent is effective when it is not associated with the HSA variant.

In some embodiments, the frequency of dosing at which the agent is effective when it is associated with the HSA variant is decreased by about 4 hours, 6 hours, 8 hours, 12 hours, 1 day, 2 days, 3 days, 4 days, 5 days, 6 days, 7 days, 10 days, 2 weeks, 3 weeks, or 4 weeks compared with the frequency at which the agent is effective when it is not associated with the HSA variant.

The improvement in the properties of the agent can be assessed relative to any appropriate control. For example, the improvement in the properties of an agent that is associated with an HSA variant can be assessed by comparing the properties of the agent that is associated with the HSA variant with the properties of the agent when it is not in an association with the HSA variant. Alternatively, the improvement in the properties of an agent that is associated with an HSA variant can be assessed by comparing the properties of the agent that is associated with the HSA variant with the properties of the agent when it is in an association with a corresponding native serum albumin polypeptide.

The agent can be another protein, e.g., a heterologous protein. In some embodiments, the agent is a diagnostic agent. In some embodiments, the agent is a therapeutic agent. For example, a protein that comprises an HSA variant can be used to extend the PK of a systemically administered therapeutic agent. The heterologous protein can be, for example, a therapeutic protein or a diagnostic protein. The serum albumin polypeptide with altered FcRn binding properties or a domain thereof (e.g., domain III) can be associated with (e.g., attached covalently to) the therapeutic protein, or to an active fragment or variant of the therapeutic protein. The variant serum albumin or a domain thereof can be in the same polypeptide chain as is at least a component of the therapeutic protein.

An HSA variant can be associated with another agent, e.g., a therapeutic agent or a diagnostic agent. The other agent (e.g., therapeutic agent) can be an entire protein (e.g., an entire therapeutic protein) or a biologically active fragment thereof. The activity of the agent (e.g., therapeutic agent) can be evaluated in an appropriate in vitro or in vivo assay for the agent's activity. In general, the activity of the agent fused to an HSA variant is not reduced, for example, by more than 50%, by more than 40%, by more than 30%, by more than 20%, by more than 10%, by more than 5%, or by more than 1% compared with the activity of the agent when it is not in association with the agent. Examples of methods for assessing the activity of certain agents are provided herein.

In some embodiments, the HSA variant is attached to the agent by one or more covalent bonds to form a variant serum albumin fusion molecule. Any agent that can be linked to an HSA variant described herein can be used as the agent in a variant serum albumin fusion molecule. The agent can be a therapeutic or diagnostic agent. For example, the agent can be any polypeptide or drug known to one of skill in the art.

In some embodiments, an agent (e.g., therapeutic or diagnostic agent) is associated with an HSA variant, but the agent is not fused to the HSA variant. In such embodiments, the agent can be associated with the HSA variant by any means known in the art. For example, the agent can be conjugated to a moiety that is capable of binding the HSA variant. In some embodiments, the moiety is an albumin binding protein. In some embodiments the moiety is a fatty acid. In embodiments wherein the agent is not fused to the HSA variant, the agent can be administered before, after, or concurrently with the HSA variant. In some embodiments, the agent is administered concurrently with the HSA variant. In some embodiments, the agent is administered at the same frequency as is the HSA variant. In some embodiments, the agent is administered more or less frequently than the HSA variant.

In some embodiments, the agent is a polypeptide consisting of at least 5, for example, at least 10, at least 20, at least 30, at least 40, at least 50, at least 60, at least 70, at least 80, at least 90 or at least 100 amino acid residues. The agent can be derived from any protein for which an improved property is desired, e.g., an increase in serum levels and/or serum half-life of the agent; or a modified tissue distribution and/or tissue-targeting of the agent.

In some embodiments, the agent is a cytokine or a variant thereof. Generally, a cytokine is a protein released by one cell population that acts on another cell as an intercellular mediator. Examples of such cytokines include lymphokines, monokines, and traditional polypeptide hormones. Specific examples include: interleukins (ILs) such as IL-1 (IL-1α and IL1β), IL-2, IL-3, IL-4, IL-5, IL-6, IL-7, IL-8, IL-9, IL-10, IL-11, IL-12; IL-15, IL-18; a tumor necrosis factor such as TNF-alpha or TNF-beta; growth hormone such as human growth hormone (HGH); somatotropin; somatrem; N-methionyl human growth hormone, and bovine growth hormone; parathyroid hormone; thyroxine; insulin; proinsulin; insulin-like growth factors, such as insulin-like growth factors-1, -2, and -3 (IGF-1; IGF-2; IGF-3); proglucagon; glucagon and glucagon-like peptides, such as glucagon-like peptide-1 and -2 (GLP-1 and GLP-2); exendins, such as exendin-4; gastric inhibitory polypeptide (GIP); secretin; pancreatic polypeptide (PP); nicotinamide phosphoribosyltransferase (also known as visfatin); leptin; neuropeptide Y (NPY); interleukin IL-1Ra, including (N140Q); ghrelin; orexin; adiponectin; retinol-binding protein-4 (RBP-4); adropin; relaxin; prorelaxin; neurogenic differentiation factor 1 (NeuroD1); glicentin and glicentin-related peptide; cholecystokinin (previously known as pancreozymin); glycoprotein hormones such as follicle stimulating hormone (FSH), thyroid stimulating hormone (TSH), and luteinizing hormone (LH); hepatic growth factor; fibroblast growth factors (FGF) such as FGF-19, FGF-21 and FGF-23; prolactin; placental lactogen; tumor necrosis factor-alpha and -beta; mullerian-inhibiting substance; gonadotropin-associated peptide; luteinizing-hormone-releasing hormone (LHRH); inhibin; activin; vascular endothelial growth factor; integrin; thrombopoietin (TPO); growth factors (e.g., platelet-derived growth factor, PDGF and its receptor, EGF and its receptor, nerve growth factors, such as NGF-beta and its receptor, and KGF, such as palifermin, and its receptor); platelet-growth factor (PGF); transforming growth factors (TGFs) such as TGF-alpha and TGF-beta; osteoinductive and growth and differentiation factors, such as osteocalcin, BMP-2, BMP-4, BMP-6 and BMP-7; interferons such as interferon-alpha, beta, and -gamma, including interferon-alpha2B; colony stimulating factors (CSFs) such as macrophage-CSF (M-CSF); granulocyte-macrophage-CSF (GM-CSF); and granulocyte-CSF (G-CSF); erythropoietin (EPO); darbepoeitin alfa; tissue plasminogen activator (TPA) or alteplase; tenecteplase; dornase alfa; entanercept; calcitonin, oxyntomodulin; glucocerebrosidase; arginine deiminase, Arg-vasopressin, natriuretic peptides, including A-type natriuretic peptide; B-type natriuretic peptide, C-type natriuretic peptide and Dendroapsis natriuretic peptide (DNP); gonadotropin-releasing hormone (GnRH); endostatin; angiostatin, including (N211Q); Kiss-1; hepcidin; oxytocin; pancreatic polypeptide; calcitonin gene-related protein (CGRP); parathyroid hormone (PTH); adrenomedulin; delta-opioids; κ-opioids; mu-opioids; deltorphins; enkephalins; dynorphins; endorphins; CD276, including (B7-H3); ephrin-B1; tweak-R, cyanovirin, including cyranovirin-N; gp41 peptides; 5-helix protein; prosaptide; apolipoprotein A1; BDNF; brain-derived neural protein; CNTF (Axokine®); antithrombin III; FVIII A1 domain; Kringle-5; Apo A-1 Milano; Kunitz domains; vWF A1 domain; Peptide YY, including PYY1-36 and PYY3-36; urate oxidase; and other polypeptide factors including LIF and kit ligand (KL).

In one embodiment, the agent is BMP peptide analogue (e.g., THR-184, Thrasos Therapeutics, Inc., Laval, QC, Canada).

Other agents suitable for use with an albumin such as an HSA variant have been described in the art, for example, see PCT/US2012/065733.

Production

A variety of molecular biology techniques can be used to design nucleic acid constructs encoding a protein that includes a serum albumin or a domain thereof. The coding sequence can include, e.g., a sequence encoding a protein described herein, a variant of such sequence, or a sequence that hybridizes to such sequences. An exemplary coding sequence for mammalian expression can further include an intron. Coding sequences can be obtained, e.g., by a variety of methods including direct cloning, PCR, and the construction of synthetic genes. Various methods are available to construct useful synthetic genes, see, e.g., the GeneArt® GeneOptimizer® from Life Technologies, Inc. (Carlsbad, Calif.), Sandhu et al. (2008) In Silico Biol 8: 0016; Gao et al. (2004) Biotechnol Prog, 20: 443-8.; Cai et al. (2010) J Bioinformatics Sequence Analysis 2: 25-29; and Graf et al. (2000), J Virol 74: 10822-10826.

The coding sequence generally employs one or more codons according to the codon tables for eukaryotic or prokaryotic expression. A coding sequence can be generated with specific codons (e.g., preferred codons) and/or one or more degenerate codons using methods known in the art.

A protein described herein, such as a protein containing a serum albumin domain described herein, can be expressed in bacterial, yeast, plant, insect, or mammalian cells.

Exemplary mammalian host cells for recombinant expression include Chinese Hamster Ovary (CHO cells) (including dhfr− CHO cells, described in Urlaub and Chasin (1980) Proc Natl Acad Sci USA 77:4216-4220, used with a DHFR selectable marker, e.g., as described in Kaufman and Sharp (1982) Mol Biol 159:601 621, lymphocytic cell lines, e.g., NS0 myeloma cells and SP2 cells, COS cells, K562, and a cell from a transgenic animal, e.g., a transgenic mammal. For example, the cell can be a mammary epithelial cell.

Coding nucleic acid sequences can be maintained in recombinant expression vectors that include additional nucleic acid sequences, such as a sequence that regulate replications of the vector in host cells (e.g., origins of replication) and a selectable marker gene. The selectable marker gene facilitates selection of host cells into which the vector has been introduced. Exemplary selectable marker genes appropriate for mammalian cells include the dihydrofolate reductase (DHFR) gene (for use in dhfr− host cells with methotrexate selection/amplification) and the neo gene (for G418 selection).

Within the recombinant expression vector, the coding nucleic acid sequences can be operatively linked to transcriptional control sequences (e.g., enhancer/promoter regulatory elements) to drive high levels of transcription of the genes. Examples of eukaryotic transcriptional control sequences include the metallothionein gene promoter, promoters and enhancers derived from eukaryotic viruses, such as SV40, CMV, adenovirus and the like. Specific examples include sequences including a CMV enhancer/AdMLP promoter regulatory element or an SV40 enhancer/AdMLP promoter regulatory element.

An exemplary recombinant expression vector also carries a DHFR gene, which allows for selection of CHO cells that have been transfected with the vector using methotrexate selection/amplification. The selected transformant host cells are cultured to allow for expression of the protein.

An adenovirus system can also be used for protein production. By culturing adenovirus-infected non-293 cells under conditions in which the cells are not rapidly dividing, the cells can produce proteins for extended periods of time. For example, BHK cells are grown to confluence in cell factories, and exposed to the adenoviral vector encoding the secreted protein of interest. The cells are then grown under serum-free conditions, which allows infected cells to survive for several weeks without significant cell division. In another method, adenovirus vector-infected 293 cells can be grown as adherent cells or in suspension culture at relatively high cell density to produce significant amounts of protein (See Gamier et al. (1994) Cytotechnol 15:145-55 and Liu et al. (2009) J Biosci Bioeng, 107:524-529. The expressed, secreted heterologous protein can be repeatedly isolated from the cell culture supernatant, lysate, or membrane fractions depending on the disposition of the expressed protein in the cell. Within the infected 293 cell production protocol, non-secreted proteins can also be effectively obtained.

Insect cells can be infected with recombinant baculovirus, commonly derived from Autographa californica nuclear polyhedrosis virus (AcNPV) according to methods known in the art. Recombinant baculovirus can be produced through the use of a transposon-based system described by Luckow et al. (1993, J Virol 67:4566-4579). This system, which utilizes transfer vectors, is commercially available in kit form (Bac-to-Bac® kit; Life Technologies, Rockville, Md.). An exemplary transfer vector (e.g., pFastBac1™ Life Technologies) contains a Tn7 transposon to transfer the DNA encoding the protein of interest into a baculovirus genome maintained in E. coli as a bacmid (e.g., Condreay et al. (2007) Curr Drug Targets 8:1126-1131). In addition, transfer vectors can include an in-frame fusion with DNA encoding a polypeptide extension or affinity tag as disclosed above. Using techniques known in the art, a transfer vector containing nucleic acid sequence encoding a variant serum albumin fusion is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodoptera frugiperda cells, such as Sf9 cells. Recombinant virus that expresses a protein containing a serum albumin domain is subsequently produced. Recombinant viral stocks are made by methods commonly used the art.

For protein production, the recombinant virus is used to infect host cells, typically a cell line derived from the fall armyworm, Spodoptera frugiperda (e.g., Sf9 or Sf21 cells) or Trichoplusia ni (e.g., High Five™ cells (BTI-TN-5B1-4); Invitrogen, Carlsbad, Calif.); for example, see U.S. Pat. No. 5,300,435. Serum-free media are used to grow and maintain the cells. Suitable media formulations are known in the art and can be obtained from commercial suppliers. The cells are grown up from an inoculation density of approximately 2-5×10⁵ cells to a density of 1-2×10⁶ cells, at which time a recombinant viral stock is added at a multiplicity of infection (MOI) of 0.1 to 10, more typically near 3. Procedures used are generally known in the art.

Other higher eukaryotic cells can also be used as hosts, including plant cells and avian cells. Agrobacterium rhizogenes can be used as a vector for expressing genes in plant cells, e.g., O'Neill et al. (2008) Biotechnol Prog 24:372-376.

Fungal cells, including yeast cells, can also be used within the present invention. Yeast species of particular interest in this regard include Saccharomyces cerevisiae, Hansenula polymorpha, Kluyveromyces lactis, Pichia pastoris, and Pichia methanotica. Transformed cells are selected by phenotype determined by the selectable marker, commonly drug resistance or the ability to grow in the absence of a particular nutrient (e.g., leucine). Production of recombinant proteins in Pichia methanolica is described, e.g., in U.S. Pat. No. 5,716,808, U.S. Pat. No. 5,736,383, U.S. Pat. No. 5,854,039, and U.S. Pat. No. 5,888,768.

The binding protein is recovered from the culture medium and can be purified. Various methods of protein purification can be employed and such methods are known in the art and described for example in Deutscher, Methods in Enzymology, 182 (1990); and Scopes, Protein Purification: Principles and Practice, Springer-Verlag, New York (2010) (ISBN: 1441928332). Purified variant serum albumin fusion proteins can be concentrated using known protein concentration techniques.

Exemplary of purification procedures include: ion exchange chromatography, size exclusion chromatography, and affinity chromatography as appropriate. For example, variant serum albumin fusion proteins can be purified with a HSA affinity matrix.

To prepare the pharmaceutical composition a variant serum albumin fusion protein is typically at least 10, 20, 50, 70, 80, 90, 95, 98, 99, or 99.99% pure and typically free of other proteins including undesired human proteins and proteins of the cell from which it is produced. It can be the only protein in the composition or the only active protein in the composition or one of a selected set of purified proteins. Purified preparations of a variant serum albumin fusion protein described herein can include at least 50, 100, 200, or 500 micrograms, or at least 5, 50, 100, 200, or 500 milligrams, or at least 1, 2, or 3 grams of the binding protein. Accordingly, also featured herein are such purified and isolated forms of the binding proteins described herein. The term “isolated” refers to material that is removed from its original environment (e.g., the cells or materials from which the binding protein is produced).

Linkers

In some embodiments described herein, an HSA variant is associated with an agent (e.g., a diagnostic or therapeutic agent), e.g., for the purpose of improving a functional property (e.g., extending the PK) of the agent. In some embodiments, the HSA variant is physically attached to the agent. The HSA variant can be directly attached to the agent or it can be attached to the agent via a linker.

In some embodiments, a heterologous protein that comprises an HSA variant and an additional agent (e.g., a diagnostic or therapeutic agent) is made using recombinant DNA techniques. In some embodiments, the HSA variant is produced (e.g., using recombinant DNA techniques) and subsequently linked to the agent, e.g., by chemical means.

A variety of linkers can be used to join a polypeptide component of an agent to domain III or a variant serum albumin. The linker can be a molecule or group of molecules (such as a monomer or polymer) that connects two molecules and optionally to place the two molecules in a particular configuration. Exemplary linkers include polypeptide linkages between N- and C-termini of proteins or protein domains, linkage via disulfide bonds, and linkage via chemical cross-linking reagents.

In some embodiments, the linker includes one or more peptide bonds, e.g., generated by recombinant techniques or peptide synthesis. The linker can contain one or more amino acid residues that provide flexibility. In some embodiments, the linker peptide predominantly includes the following amino acid residues: Gly, Ser, Ala, and/or Thr. The linker peptide should have a length that is adequate to link two molecules in such a way that they assume the correct conformation relative to one another so that they retain the desired activity. Suitable lengths for this purpose include at least one and not more than 30 amino acid residues. For example, the linker is from about 1 to 30 amino acids in length. A linker can also be, for example, 1, 2, 3, 4, 5, 6, 7, 8, 9, 10, 11, 12, 13, 14, 15, 16, 17, 18 19 and 20 amino acids in length.

Exemplary linkers include glycine-serine polymers (including, for example, (GS)n, (GSGGS)n, (GGGGS)n and (GGGS)n, where n is an integer of at least one, e.g., one, two, three, or four), glycine-alanine polymers, alanine-serine polymers, and other flexible linkers. Glycine-serine polymers can serve as a neutral tether between components. Secondly, serine is hydrophilic and therefore able to solubilize what could be a globular glycine chain. Third, similar chains have been shown to be effective in joining subunits of recombinant proteins such as single chain antibodies. Suitable linkers can also be identified from three-dimensional structures in structure databases for natural linkers that bridge the gap between two polypeptide chains. In some embodiments, the linker is from a human protein and/or is not immunogenic in a human. Thus linkers can be chosen such that they have low immunogenicity or are thought to have low immunogenicity. For example, a linker can be chosen that exists naturally in a human. In certain embodiments the linker has the sequence of the hinge region of an antibody, that is the sequence that links the antibody Fab and Fc regions; alternatively the linker has a sequence that comprises part of the hinge region, or a sequence that is substantially similar to the hinge region of an antibody. Another way of obtaining a suitable linker is by optimizing a simple linker, e.g., (Gly₄Ser)ₙ, through random mutagenesis. Alternatively, once a suitable polypeptide linker is defined, additional linker polypeptides can be created to select amino acids that more optimally interact with the domains being linked. Other types of linkers include artificial polypeptide linkers and inteins. In another embodiment, disulfide bonds are designed to link the two molecules. Other examples include peptide linkers described in U.S. Pat. No. 5,073,627, the disclosure of which is hereby incorporated by reference. In certain cases, the diagnostic or therapeutic protein itself can be a linker by fusing tandem copies of the peptide to a variant serum albumin polypeptide. In certain embodiments, charged residues including arginine, lysine, aspartic acid, or glutamic acid can be incorporated into the linker sequence in order to form a charged linker.

In another embodiment, linkers are formed by bonds from chemical cross-linking agents. For example, a variety of bifunctional protein coupling agents can be used, including but not limited to N-succinimidyl-3-(2-pyridyldithiol) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). Chemical linkers can enable chelation of an isotope. For example, C14 1-isothiocyanatobenzyl-3-methyldiethylene triaminepentaacetic acid (MX-DTPA) is an exemplary chelating agent for conjugation of radionucleotide to the antibody (see PCT WO 94/11026).

The linker can be cleavable, facilitating release of a payload, e.g., in the cell or a particular milieu. For example, an acid-labile linker, peptidase-sensitive linker, dimethyl linker or disulfide-containing linker (Chari et al. (1992) Cancer Res 52:127-131) can be used. In some embodiments, the linker includes a nonproteinaceous polymer, e.g., polyethylene glycol (PEG), polypropylene glycol, polyoxyalkylenes, or copolymers of polyethylene glycol and polypropylene glycol.

In one embodiment, the variant serum albumin fusion of the present invention is conjugated or operably linked to another therapeutic compound, referred to herein as a conjugate. The conjugate can be a cytotoxic agent, a chemotherapeutic agent, a cytokine, an anti-angiogenic agent, a tyrosine kinase inhibitor, a toxin, a radioisotope, or other therapeutically active agent. Chemotherapeutic agents, cytokines, anti-angiogenic agents, tyrosine kinase inhibitors, and other therapeutic agents have been described above, and the aforementioned therapeutic agents can find use as variant serum albumin fusion conjugates. In an alternate embodiment, the variant serum albumin fusion is conjugated or operably linked to a toxin, including but not limited to small molecule toxins and enzymatically active toxins of bacterial, fungal, plant or animal origin, including fragments and/or variants thereof. Small molecule toxins include but are not limited to calicheamicin, maytansine (U.S. Pat. No. 5,208,020), trichothene, and CC1065. In one embodiment of the invention, the variant serum albumin fusion is conjugated to one or more maytansine molecules (e.g., about 1 to about 10 maytansine molecules per antibody molecule). Maytansine can, for example, be converted to May-SS-Me which can be reduced to May-SH3 and reacted with a variant serum albumin fusion (Chari et al. (1992) Cancer Res 52: 127-131) to generate a maytansinoid-antibody or maytansinoid-Fc fusion conjugate. Another conjugate of interest comprises a variant serum albumin fusion conjugated to one or more calicheamicin molecules. The calicheamicin family of antibiotics are capable of producing double-stranded DNA breaks at sub-picomolar concentrations. Structural analogs of calicheamicin that can be used include but are not limited to γi, α₂, α₃, N-acetyl-γi, θ, alpha3, N-acetyl-11, PSAG, and gamma 11, (Hinman et al (1993) Cancer Res 53:3336-3342; Lode et al. (1998) Cancer Res 58:2925-2928) (U.S. Pat. No. 5,714,586; U.S. Pat. No. 5,712,374; U.S. Pat. No. 5,264,586; U.S. Pat. No. 5,773,001). Dolastatin 10 analogs such as auristatin E (AE) and monomethylauristatin E (MMAE) can be used in conjugates for the variant serum albumin fusions of the present invention (Doronina et al. (2003) Nat Biotechnol 21:778-84; Francisco et al. (2003) Blood 102:1458-65). Useful enzymatically active toxins include but are not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, PCT WO 93/21232. The present invention further contemplates a conjugate or fusion formed between a variant serum albumin fusion of the present invention and a compound with nucleolytic activity, for example a ribonuclease or DNA endonuclease such as a deoxyribonuclease (DNase).

In an alternate embodiment, a variant serum albumin fusion of the present invention can be conjugated or operably linked to a radioisotope to form a radioconjugate. A variety of radioactive isotopes are available for the production of radioconjugate variant serum albumin fusions. Examples include, but are not limited to, At211, I131, I125, Y90, Re186, Re188, Sm153, Bi212, P32, and radioactive isotopes of Lu.

### Screening Methods

### Assays

Binding of an HSA variant or candidate (mutated) HSA variant to FcRn can be evaluated in vitro, e.g., by surface plasmon resonance (SPR), ELISA, or other binding assay known in the art.

FcRn can be produced as a single chain molecule, e.g., in CHO cells. An exemplary method for producing single chain FcRn is described in Feng et al. (2011) Protein Expression and Purification, 79:66-71.

The half-life of a protein that includes serum albumin or a domain thereof in vivo can be evaluated in a mammal, e.g., a murine model that includes a human FcRn. See e.g., Example 3. For example, the protein that is evaluated can be a protein that includes serum albumin or a domain thereof and a therapeutic agent.

### Activity Assays

To assess the activity of an agent (e.g., a therapeutic agent) that is associated with an HSA variant as described herein, methods known in the art for testing the activity of the agent can be used.

Further illustration of the invention is provided by the following non-limiting examples.

### EXAMPLES

### Example 1

### Summary of Methods

A library of HSA variants with random changes in DIII was fused to the high affinity anti-fluorescein scFv, 4M5.3 (e.g., Boder et al. (2000) Proc Nat Acad Sci 97:10701-10795). Fluoresceinated yeast cells captured secreted 4M5.3-HSA variants on their surface. Bound HSA was labeled with soluble, single-chain hFcRn and high affinity binders selected by FACS. In the method developed by applicants, the protein of interest is fused to 4M5.3, an scFv that binds fluorescein with fM affinity. Cells are chemically conjugated to fluorescein with an NHS-PEG-fluorescein reagent, and the freely secreted protein is captured on the cell surface. Without fluoresceination, the protein is secreted to the medium.

To obtain crystals, a 1:1 complex of HSA13 and hFcRn was isolated on a gel filtration column at pH 5.5. Crystals were grown by hanging drop at pH 4.9. The structure was solved by molecular replacement using HSA and human FcRn as search models (PDB codes 1AO6 and 3M17). The final structure was refined to excellent statistics (Table 3) after manual rebuilding and refinement in Coot (Emsley et al. (2010) Acta Crystallographica. Section D. Biological Crystallography 66:486-501) and CCP4 (REFMAC5) (Murshudov et al (1997) Acta Crystallographica. Section D. Biological Crystallography 53:240-255) using TLS.

TABLE 2
ELISA affinities of immobilized HSA variants binding to scFcRn at pH 6.0 or 5.5*. The basis for the
affinity increase of V547A is not clear. Position 547 packs against Leu-532, and the smaller Ala side
chain brings Leu-532 ~1.4 Å closer in, potentially facilitating movement of the DIIIB helices.

| HSA | | | | | | | | | | | | |
|---|---|---|---|---|---|---|---|---|---|---|---|---|
| variant | V418 | T420 | V424 | N429 | M444 | A469 | T467 | E492 | E505 | V547 | A552 | K_{D} (nM) |
| HSA13 | M | A | | | | | | | G | A | | 3.0 ± 0.4 |
| HSA12 | M | A | | | | | | | | A | | 10.5 ± 0.4 |
| HSA7 | | | | | | | | | G | A | | 22.6 ± 3.0 |
| HSA21 | M | A | | | | | | | R | | | 50.1 ± 4.8 |
| HSA11 | M | A | | | | | | | G | | | 92.6 ± 9.3 |
| HSA5 | | | | | | | | | | A | | 326.1 ± 93 |
| HSA10 | M | A | | | | | | | | | | 368.1 ± 39 |
| HSA6 | | | | | | | | | G | | | 371.4 ± 50 |
| HSA16 | | | | | | | | | R | | | 395.0 ± 90 |
| HSA9 | | A | | | | | | | | | | 419.7 ± 31 |
| HSA8 | M | | | | | | | | | | | 589.8 ± 40 |
| HSA | | | | | | | | | | | | 1030.3 ± 292 |
| HSA15 | M | A | I | D | | | | | R | | | 3* |
| HSA14 | M | A | | | V | V | M | | G | | T | 50* |
| (Clone A) | | | | | | | | | | | | |
| HSA18 | | | I | | | | | | | | | 250* |
| HSA4 | | | | | | | | G | | | | >1000* |
| HSA17 | | | | | | | | | K | | | >1000* |
| HSA19 | | | | D | | | | | | | | >1000* |
| HSA20 | | | | | V | V | | | | | | >1000* |
| HSA22 | | | | | | | M | | | | | >1000* |
| HSA23 | | | | | | | | | | | T | >1000* |

For PK studies, HSA proteins were expressed in S. cerevisiae and purified by HSA affinity resin and anion exchange chromatography. Untagged or HA-tagged HSA variants were administered IV in wild type and hFcRn transgenic mice and Cynomolgus monkeys, respectively, and plasma concentrations measured over time by ELISA.

Vector Construction

The 4M5.3 S. cerevisiae display vector comprised the high affinity anti-fluorescein scFv 4M5.3 (Boder et al. supra) with an N-terminal app8 leader sequence (Rakestraw et al. (2009) Biotechnol Bioeng 103:1192-1201) and C-terminal (G₄S)₃ linker followed by cloning sites, an HA epitope tag (YPYDVPDYA; SEQ ID NO:4), and stop codon, in a variant pYC2/CT expression vector (Life Technologies) with TRP1 replacing URA3. The 4M5.3-HSA library backbone was generated by PCR amplifying the DI and DII domains of human serum albumin cDNA (NM_{—}000477.3, Origene, Rockville, Md.) into the 4M5.3 display vector to generate a 4M5.3-(G₄S)₃-DI DII-cloning site-HA fusion construct. For expression of HSA variants in yeast, mature HSA with an app8 leader sequence was expressed from an unmodified pYC2/CT vector. For in vivo studies in primates, a hemagglutinin (HA) tag was added to the N-terminus of HSA by QuikChange® mutagenesis (Agilent Technologies, Santa Clara, Calif.).

The single-chain human FcRn (schFcRn) construct comprised β2m fused to the extracellular domain of the FcRn α-chain through a (G₄S)₃ linker (SEQ ID NO:5) as described previously (e.g., see Feng et al. (2011) Prot Expression Purification 79:66-71) cloned into a modified version of the pcDNA3.1(+) vector (Life Technologies) containing an N-terminal IL-2 leader sequence (MYRMQLLSCIALSLALVTNS, SEQ ID NO:1) and C-terminal FLAG tag (DYKDDDDK, SEQ ID NO:2). To generate material for binding studies, a higher expressing vector was constructed by cloning the IL-2 leader and schFcRn into a variant of the pTT5 expression vector (NRCC) containing a C-terminal FLAG/His tag (DYKDDDDKNSAHHHHHHHH, SEQ ID NO:3). A cDNA encoding mouse single-chain FcRn was also synthesized and cloned into the pTT5-FLAG/His vector as above except that the IL-2 leader was replaced with the native murine β2m sequence. For crystallography material, the expression cassette was subcloned into pLVX (Clontech, Mountain View, Calif.). HSA and FcRn point mutants were generated by Quikchange mutagenesis or overlap extension PCR according to standard techniques.

Single-Chain FcRn Expression and Purification

FLAG-tagged schFcRn was harvested from the supernatant of transiently transfected Freestyle-CHO cells (Life Technologies, Woburn, Mass.) grown for 7 days. Protein was purified on an M2 anti-FLAG affinity column (Sigma, St. Louis, Mo.), eluted with 100 mM glycine-HCl, pH 3.5 and immediately neutralized with 1/10^{th} volume 1 M Tris-HCl, pH 8.0 before buffer exchanging into PBS, pH 7.4. For yeast selections, the protein was biotinylated using Sulfo-NHS-LC-biotin reagent (Pierce, Rockford, Ill.) and excess biotin removed through several rounds of concentration and dilution into PBS using 10 kDa Amicon Ultra-15 spin filters (Millipore).

FLAG/His-tagged schFcRn used in ELISA and SPR studies was harvested from the supernatant of transiently transfected HEK293-6E cells grown for 7 days. Proteins were purified with Ni-NTA affinity resin (Life Technologies) pre-equilibrated with 50 mM NaH₂PO₄, 500 mM NaCl, pH 8.0, eluted in 50 mM NaH₂PO₄, 500 mM NaCl, 250 mM imidazole, pH 8.0 and buffer exchanged into PBS, pH 7.4. Additional polishing was performed as needed on a Superdex® 75 column (GE Healthcare, Piscataway, N.J.) in PBS.

For crystallography material, the pLVX-schFcRn plasmid was used to make lentivirus that was used to transduce HEK293-6E cells. His-tagged schFcRn was harvested from the supernatant of cells grown in a 50 l BIOSTAT® CultiBag (Sartorius, Bohemia, N.Y.) system with a 25 l working volume. Protein was purified on a high performance Ni-Sepharose™ column (GE Healthcare) pre-equilibrated with 20 mM Tris, 1 M NaCl, pH 8.5. The column was washed with 20 mM Tris, 1 M NaCl, 10 mM imidazole, pH 8.5. Protein was eluted with 20 mM Tris, 1 M NaCl, 250 mM imidazole, pH 8.5 then buffer exchanged into PBS, pH 7.4. Protein purity for all scFcRn constructs was assessed by SDS-PAGE and concentration determined by absorbance at 280 nm.

Library Generation and Induction

Error prone mutagenesis and library generation in yeast was performed as described in the art (see Chao et al. (2006) Nature Protocols 1:755-768. Briefly, the DIII domain of human serum albumin was amplified using primers that added ˜50 bp of homology with the flanking sequences of the DII domain and C-terminal vector. The 4M5.3-HSA library backbone was linearized and mixed with the PCR insert and precipitated using Pellet Paint (EMD Millipore). The DNA pellet was resuspended in a BJ5α (ATCC) suspension prepared as described in the art (Chao et al. supra) then electroporated and grown in SDCAA media (2% glucose, 0.67% yeast nitrogen base, 0.5% casein amino acids, 0.54% Na₂HPO₄, 0.86% NaH₂PO₄.H20, and 1× penicillin/streptomycin) supplemented with 40 mg/l uracil to select for transformants.

Secretion and capture of the 4M5.3-HSA library was performed similarly to previously described protocols (Rakestraw et al. (2006) Biotechnol Prog 22:1200-1208). Briefly, cells were grown in SDCAA+uracil at 30° C. and induced in yeast extract-peptone-glycerol (YPG) medium at an OD₆₀₀ of ˜5 at 20° C. for 6-8 hours. Cells were washed three times in sodium bicarbonate buffer (4.2% NaHCO₃, 0.034% Na₂CO₃, pH 8.4), and labeled with 100 mg/ml NHS-PEG_{3500Da}-Fluorescein (Creative PEGWorks, Winston Salem, N.C.) for 30 minutes at RT. Labeled cells were washed three times with PBS+0.1% BSA then resuspended in YPG medium with 20% (w/v) PEG (35 kDa, Sigma) and incubated in a static culture at 20° C. for 14-16 hours. The expressed library was then resuspended in PBS-BSA with 100 nM FITC-Dextran (Sigma) to bind free 4M5.3-HSA and washed in PBS-BSA several times.

FACS Selections

Libraries were simultaneously labeled for schFcRn binding and HSA display level. For selection rounds 1-4, 20 nM biotinylated schFcRn-FLAG was pre-loaded onto 5 nM streptavidin-APC (Life Technologies) to form tetramers prior to incubating with the cells. For rounds 5-7, monomeric biotinylated schFcRn was incubated with the cells followed by detection with NeutrAvidin-DyLight®-650 (1:5000, Pierce). In all rounds, cells were labeled for display with an anti-HA primary antibody (1:1000, Sigma) followed by a PE-Cy7 conjugated anti-mouse secondary (1:500, Santa Cruz, Dallas, Tex.). All labeling and wash steps were performed in PBS+0.1% fish gelatin (Sigma), pH 5.6. Libraries were run on a FACSAria III cell sorter and cells with high schFcRn binding (APC signal) relative to display (PE-Cy7 signal) were selected and re-grown in SDCAA+uracil with citrate buffer, pH 4.5. After selection rounds 3-7, DNA was isolated from the enriched yeast using the Zymoprep yeast plasmid miniprep II kit (Zymo Research, Irvine, Calif.) and transformed into XL-1 Blue competent cells. 8-12 clones from each round were then miniprepped (Qiagen, Valencia, Calif.) and their DIII domain sequenced (Genewiz).

HSA Expression & Purification

HSA plasmids were transformed into BJ5α yeast using the EZ-yeast transformation kit (Zymo Research, Irvine, Calif.) and plated on SDCAA+40 mg/1 tryptophan (SD-Trp). Transformed colonies were inoculated into liquid SD-Trp media and grown in 50-1000 ml shake-flask cultures at 30° C. with shaking to an OD₆₀₀>5, then induced in YPG medium at 20° C. for 48 hours. Cells were then pelleted and the supernatant filtered. For purification, the supernatant was loaded onto a CaptureSelect® HSA affinity column (Life Technologies, BAC) equilibrated in PBS, pH 7.4. The column was washed with PBS and bound HSA protein eluted with 20 mM Tris, 2 M MgCl₂, pH 7.4. Purified proteins were buffer exchanged into PBS, pH 7.4.

For in vivo studies, HSA variants were further purified on a Poros®HQ anion exchange column (Life Technologies, Grand Island, N.Y.) to remove endotoxin. Prior to loading, the column was equilibrated with 25 mM Tris, 50 mM NaCl, pH 7.5 and the proteins were adjusted to pH 7.5 and an equivalent tonicity of 50 mM NaCl. Bound HSA variants were eluted with a linear gradient of 0-0.6 M NaCl. Eluates were dialyzed into PBS, pH 7.4 and concentrated to 1 and 5 mg/ml. Protein purity was assessed by SDS-PAGE and concentration determined by absorbance at 280 nm.

Crystallization & Structure Determination

For crystallization, the HSA13/scFcRn complex was formed by mixing HSA13 (4.9 mg/ml) and His-tagged schFcRn (0.5 mg/ml) in a 1:1.5 molar ratio and dialyzing overnight at 4° C. in 20 mM MES, 50 mM NaCl, pH 5.5. A 1:1 complex was isolated on a Superdex® (S200) gel filtration column (GE Healthcare, Piscataway, N.J.) equilibrated in the same pH 5.5 buffer. Complex stoichiometry was confirmed by SEC-MALS using a Zenix®-300 column (Sepax, Newark, Del.) and a MiniDAWN® Treos® static light scattering instrument with an Optilab® T-Rex refractive index detector (Wyatt Technology, Santa Barbara, Calif.).

Crystals were obtained by hanging drop vapor diffusion at 293° K. Preliminary micro-crystals grew within 2 weeks in 2 M ammonium sulfate, 0.1 M sodium acetate, pH 4.6, using a protein concentration of 13 mg/ml. To obtain crystals suitable for data collection, a seed stock was prepared from the micro-crystals and subsequently used for crystal optimization by microseeding. Diffraction quality crystals grew within 3-4 weeks in 1.7 M ammonium sulfate, 0.1 M sodium citrate, pH 4.9, using a 5:4:1 ratio of precipitant:protein:seed stock. Prior to data collection, crystals were cryo-protected in mother liquor containing 20% glycerol and flash frozen in liquid nitrogen.

Data were collected at 0.9782 Å and 100° K at beamline CMCF-08ID of the Canadian Light Source (CLS, Saskatoon, Canada) using a MARCCD detector. Images were processed with XDS and XSCALE (Kabsch (2010) Acta Crystallographica. Section D, Biological Crystallography 66:125-132). Crystals belong to the space group P21212 with two complexes per ASU and 58% solvent. A dataset extending to 2.4 Å was used for structure determination (Table 3).

The structure was solved by molecular replacement with the program PHASER (McCoy et al. (2007) J Appl Crystallography 40:658-674) using structures of HSA and human FcRn (PDB accessions 1AO6 and 3M17) as search models. Subsequent model rebuilding and refinement were performed in several cycles using Coot (Emsley et al. (2010) Acta Crystallographica. Section D. Biological Crystallography 66:486-501) and CCP4 (Murshudov et al. (1997) Acta Crystallographica. Section D. biological Crystallography 53:240-255). TLS refinement (using REFMAC5, CCP4) (Murshudov et al., supra) was applied, resulting in lower R-factors and higher quality electron density maps. Statistics of the final structure are listed in Table 3. The final model was refined to a R_{work}/R_{free} value of 21.2%/25.2%. The Ramachandran plot shows 93.3%, 6.6% and 0.1% of all residues are in the most favored, additionally allowed and generously allowed regions, with no residues in the disallowed region. The model contains two copies in the asymmetric unit which differ with a Cα RMSD of 0.35 Å. One complex (Chains A, B and C) was primarily used for structural analyses since the electron density is better defined for the side chains. Contact maps and buried surface area values were calculated using the Protein Interfaces, Surfaces, and Assemblies (PISA) server (Krissinel and Henrick (2007) J Mol Biol 372:774-797). Structural figures were prepared using PyMOL (Schrödinger, Cambridge, Mass.). Atomic coordinates and structure factor amplitudes for the structures have been deposited in the Protein Data Bank (PDB ID code 4K71).

Affinity Measurements

Affinity ELISAs.

Purified HSA variants at 2 μg/ml in PBS, pH 7.4 were immobilized in 96 well flat-bottomed EIA plates (Corning) at 4° C. overnight. Coated wells were blocked with 300 μl PBS+5% fish gelatin, pH 7.4 for 2 hours then washed 3 times with 300 μl PBS+0.05% Tween-20, pH 6.0. 100 μl of FLAG/His-tagged schFcRn in PBS+1% fish gelatin+0.1% Tween-20, pH 6.0 was added to wells at a range of concentrations and incubated for 2 h at RT. Wells were washed as above before adding 100 μl of anti-FLAG-HRP (1:1000, Sigma) in PBS+1% fish gelatin+0.1% Tween-20, pH 6.0. After 20 min at RT, wells were washed as above and 50 μl TMB substrate (Pierce) added. Color development was stopped after one minute with 50 μl 2 M sulfuric acid and the signal measured as A₄₅₀-A₅₅₀ on a Spectramax M5 plate reader (Molecular Devices). The background signal measured on wells with no immobilized HSA was subtracted from each reading and the resulting values fit to an equilibrium K_{D} model (Abs=(Max*[FcRn]/([FcRn]+K_{D})). All samples were run in triplicate.

Surface Plasmon Resonance (SPR).

SPR studies were performed on a Reichert SR7000DC Spectrometer. HSA, schFcRn, or scmFcRn were immobilized on 500-kDa carboxymethyl dextran chips (Reichert) via standard amine coupling with 20 μg/ml protein in 10 mM sodium acetate, pH 4.5. Serial dilutions of HSA or scFcRn variants in PBS+0.005% Tween-20, pH 6.0 or 7.4, were injected at 25° C. with a 50 μl/minute flow rate and data collected over time. Reference cell values and signal from buffer injection controls were subtracted, and the sensogram traces were fit to a 1:1 kinetic binding model using Scrubber2 software (BioLogic, Campbell, Australia) to calculate kₐ, k_{d}, and K_{D} values. For studies examining the effect of fatty acid (FA) loading, sodium palmitate or sodium oleate (Sigma) were dissolved in ddH₂0 at 70° C., then added to dilapidated HSA (Sigma) in PBS to a final concentration of 2 mM FA/200 μM HSA. The FA:HSA mixture was incubated at 37° C. for 60 minutes, then filtered through a 0.2 μm membrane and buffer exchanged into PBS, pH 6.0. FA-loaded HSA was flowed over a schFcRn-coated chip at a 10 μM concentration and the binding signal recorded over time.

In Vivo Studies

Mouse.

PK studies were performed in C57BL/6J mice and FcRn^{(-/-)} C57BL/6J mice homozygous for a transgene bearing the human gene (Jackson Laboratories strain 4919). Eight to nine week old female mice weighing 16-19 g were divided into groups (N=3) with approximately equal average weights. Mice were anesthetized using isoflurane and injected intravenously in the retro orbital venus plexus with 50 μl of HSA or HSA variants at 1 mg/ml in PBS, pH 7.4. At selected times post-injection, 25 μl of blood was collected via tail nick and mixed with citrate phosphate dextrose (CPD) at a 1:1 (v/v) ratio. Samples were centrifuged at 4° C. for 5 minutes at 14,000 rpm, plasma collected, and immediately frozen and stored at −80° C. until analyzed.

Cynomolgus.

PK studies were performed in male monkeys weighing 4.5-7.2 kg (Sinclair). Pre-bleeds were drawn from all potential animals and screened for the presence of pre-existing antibodies against HA-tagged HSA molecules by ELISA. Selected animals were divided into groups with approximately equal average weights (N=6 for 1 mg/kg HA-HSA, N=2 for all other groups) and injected with a 1 mg/kg or 5 mg/kg IV bolus dose of HA-HSA or HA-HSA7 in PBS. At selected times, blood samples were drawn from each animal via direct venipuncture of the femoral vein into a 3 ml K3-EDTA tube. The blood samples were centrifuged at 4° C., 3000 RPM for 15 minutes and the plasma collected and stored at −70° C.

PK Assays.

For mouse samples EIA plates (Corning) were coated overnight with anti-HSA antibody (Abcam, Cambridge, England) at 2 μg/ml in carbonate buffer. Plates were blocked for 2 hours with 300 μl PBS+5% fish gelatin, pH 7.4 then washed 3 times with 300 μl PBS+0.1% Tween-20, pH 7.4. Plasma samples were diluted 1:10 in PBS then mixed with 10% C57BL/6J female mouse plasma:CPD (Bioreclamation) in PBS and added to wells at final dilutions of 1:40, 1:400, 1:400 and 1:4000. A standard curve was included on each plate with purified HSA diluted in 10% mouse plasma:CPD. Plates were incubated at room temperature (RT) for 2 hours then washed as above. 100 μl of anti-HSA-HRP (Bethyl Laboratories, Montgomery, Tex.) was added to each well in PBS+0.1% FG+0.1% Tween-20, pH 7.4 and incubated for 60 minutes at RT. Plates were washed as above and signal developed with 50 μl TMB substrate for 1 minute before stopping with 2 M sulfuric acid. The signal was measured on a SpectraMax® M5 plate reader as A₄₅₀-A₅₅₀ and the background signal from wells with no plasma sample subtracted. The standard curve on each plate was fitted to an equilibrium K_{D} model (Abs=(Max*[HSA])/([HSA]+K_{D})) and the plasma HSA concentration calculated at each time point as [HSA]=Abs*Dilution*K_{D}/(Max−Abs) using the dilutions that fell within the linear range of the standard curve. Anti-HSA immune responses in the animals were measured by titrating mouse plasma samples on HSA coated plates and detecting bound antibody with HRP conjugated anti-mouse-IgG antibody (1:1000, Rockland).

For primate samples ELISAs were performed as described above for mice except that plates were coated with anti-HA capture antibody (Sigma, St. Louis, Mo.) at 1 μg/ml in PBS and sample dilutions were made in 10% male Cynomologus monkey K3-EDTA plasma (Bioreclamation, Liverpool, N.Y.) in PBS at final dilutions of 1:10, 1:100, 1:1000, and 1:10,000.

PK Analysis.

All PK values other than terminal half-lives were computed using in-house software implementing standard non-compartmental analysis (NCA) methods (Gabrielsson and Weiner (2007) Pharmacokinetic and Pharmacodynamic Data Analysis: Concepts and Applications, 4^{th} ed (Swedish Pharmaceutical Press: Stockholm). Terminal half-lives were computed by fitting to a bi-exponential model using the NLINFIT function in MATLAB 2010a. Statistical comparisons for PK data were computed using an unpaired two-tailed t-test for populations with unequal variances as implemented by the TTEST2 function in MATLAB 2010a.

### Example 2

### Isolation of High-Affinity HSA Variants

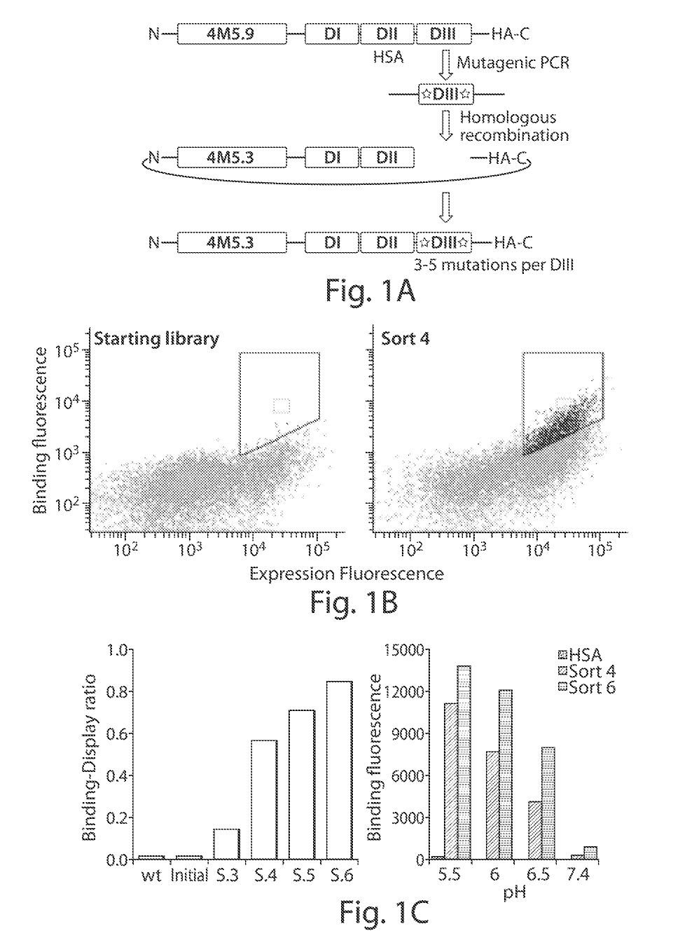

HSA is comprised of three structurally related domains (DI-DIII), each composed of subdomains A and B connected by long intradomain loops, with DIII reportedly being key for FcRn interaction. To isolate variants with increased affinity, a modified yeast secretion and capture system was used in which HSA is expressed as a fusion with the high affinity anti-fluorescein scFv 4M5.3 and captured on the surface of secreting cells by binding to fluorescein chemically conjugated to the cell surface (see Rakestraw et al (2006) Biotechnol Prog 22:1200-1208). An HSA library with random changes in DIII introduced through error-prone PCR was displayed on yeast that were sorted by FACS (FIG. 1A and FIG. 1B) for increased binding to soluble, single-chain hFcRn (schFcRn) at endosomal pH. Progressively higher binding was observed starting in round 3, while maintaining pH dependence. After 6 sorts, the pool began to exhibit undesirable affinity for schFcRn at pH 7.4 (FIG. 1C). Sequence analysis of clones from sorts three through seven showed successive enrichment of variants at four positions (V418, T420, E505 and V547), with additional mutations found in some clones (Table 6).

TABLE 6
Selected changes, sampled after successive sorts. Shown are changes
that appeared more than once in a sorted population. Clone A was
a “haplotype” consisting of V418M/T420A/M446V/A449V/
T467M/E505G/A552T. Sort 7 had collapsed into a single clone both by
FACS behavior and sequence analysis.

| | Sort (sequenced clones) | | | | |
|---|---|---|---|---|---|
| Change | 3 (11) | 4 (8) | 5 (11) | 6 (10) | 7 (8) |
| N391D | 2 | 1 | 0 | 0 | 0 |
| K402E | 1 | 2 | 0 | 0 | 0 |
| V418M | 4 | 1 | 9 | 10 | 8 |
| T420A | 3 | 1 | 8 | 10 | 8 |
| V424I | 1 | 2 | 4 | 4 | 8 |
| N429D | 2 | 1 | 2 | 4 | 8 |
| V462M | 2 | 0 | 1 | 0 | 0 |
| Clone A | 0 | 0 | 3 | 4 | 0 |
| E492G | 0 | 3 | 1 | 0 | 0 |
| E501V | 2 | 1 | 1 | 0 | 0 |
| E505G/K/ | 3 (G) | 5 (G) | 10 (7G, 2K, 1R) | 10 (6G, 3R, 1K) | 8 (R) |
| R | | | | | |
| V547A | 6 | 6 | 5 | 2 | 0 |
| K545E | 0 | 1 | 2 | 1 | 0 |

### Example 3

### Effect of HSA Mutations on hFcRn Affinity

The effect of single changes and combinations on hFcRn affinity was analyzed by ELISA and SPR at both pH 6.0 and 7.4 (Table 1 and Table 2). Single mutations increased hFcRn affinity 2-3 fold as measured by ELISA, with significantly higher affinities obtained in combinations, suggesting that each change was acting independently. The highest pH 6.0 affinity variant, HSA13, includes 4 mutations (V418M, T420A, E505G, V547A) and has a K_{D} of 3 nM at pH 6.0, compared to a >1 μM K_{D} for wild-type HSA.

TABLE 1
Kinetic data for selected HSA variants binding to immobilized single-chain
hFcRn at pH 6.0 and 7.4 as measured by surface plasmon resonance.

| HSA | kₐ | pH 6.0 | K_{D} | kₐ | pH 7.4 | K_{D} | Fold |
|---|---|---|---|---|---|---|---|
| variant | (M⁻¹s⁻¹) | k_{d} (s⁻¹)* | (nM) | (M⁻¹s⁻¹) | k_{d} (s⁻¹) | (μM) | increase |
| HSA | 6.5 × 10³ | 3.6 × 10⁻² | 5500 | NM | NM | — | — |
| HSA5 | 5.4 × 10³ | 2.4 × 10⁻³ | 440 | 1.0 × 10³ | 1.3 × 10⁻² | 130 | 300 |
| HSA7 | 1.4 × 10⁴ | 9.1 × 10⁻⁴ | 64 | 3.1 × 10³ | 1.1 × 10⁻¹ | 34 | 530 |
| HSA11 | 1.1 × 10⁴ | 2.6 × 10⁻³ | 240 | 6.7 × 10³ | 9.5 × 10⁻² | 14 | 58 |
| HSA13 | 2.7 × 10⁴ | 2.2 × 10⁻⁴ | 8.2 | 7.5 × 10³ | 5.7 × 10⁻² | 7.6 | 930 |

NM, not meaningful.
Data are from curves fitted by the supplied software.

### Example 4

### Overall Structure of the hFcRn-HSA Complex

Complexes of hFcRn/HSA and of hFcRn/HSA13 were purified. However, crystals were obtained only with HSA13, at pH 4.9. The structure of this complex was solved by molecular replacement to a 2.4 Å resolution (Table 3). Applicant's publication, Schmidt et al. (2013) StructureNov 5; 21(11):1966-78; doi: 10.1016/j.str.2013.08.022. Epub 2013 Oct. 10; incorporated herein in its entirety) provides representative stereo views of the 2Fo-Fc electron density maps for HSA13/hFcRn around hFcRnαW59 and hFcRnαW53.

TABLE 3
Crystallographic data collection and refinement statistics. *Values in
parentheses are for highest-resolution shell.

| | HSA13/hFcRn complex |
|---|---|
| Data collection | |
| Space group | P2₁2₁2 |
| Cell dimensions | |
| a, b, c (Å) | 127.89, 203.54, 100.56 |
| α, β, γ (°) | 90, 90, 90 |
| Resolution (Å) | 108.29-2.40 (2.59-2.40)* |
| No. of Reflections (Unique) | 102919 (20735) |
| R_{sym} or R_{merge} | 6.3 (67.0) |
| I/σI | 18.7 (2.3) |
| Completeness (%) | 99.7 (99.9) |
| Redundancy | 4.4 (4.4) |
| Refinement | |
| Resolution (Å) | 108.29-2.40 |
| No. reflections | 97,775 |
| R_{work}/R_{free} | 21.2/25.5 |
| Total number of atoms | |
| Protein | 15,042 |
| Water | 123 |
| Sulfate | 95 |
| B-factors | |
| Protein | 30.0 |
| Water | 27.3 |
| Sulfate | 77.5 |
| R.m.s. deviations | |
| Bond lengths (Å) | 0.013 |
| Bond angles (°) | 1.40 |
| Ramachandran plot | |
| Most favored regions (%) | 93.3 |
| Additional allowed regions (%) | 6.6 |
| Generously allowed regions (%) | 0.1 |
| Disallowed regions (%) | 0.0 |

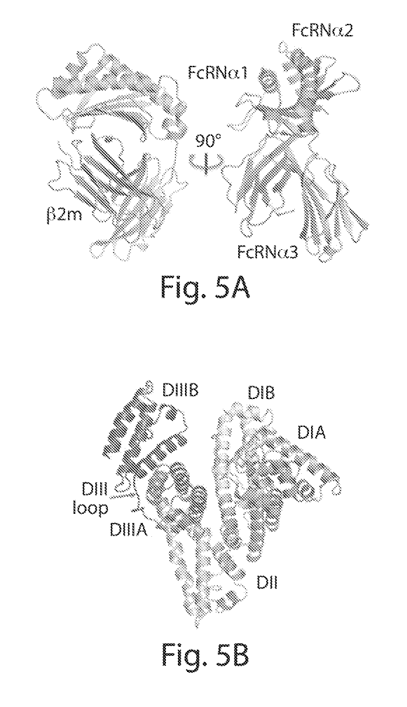

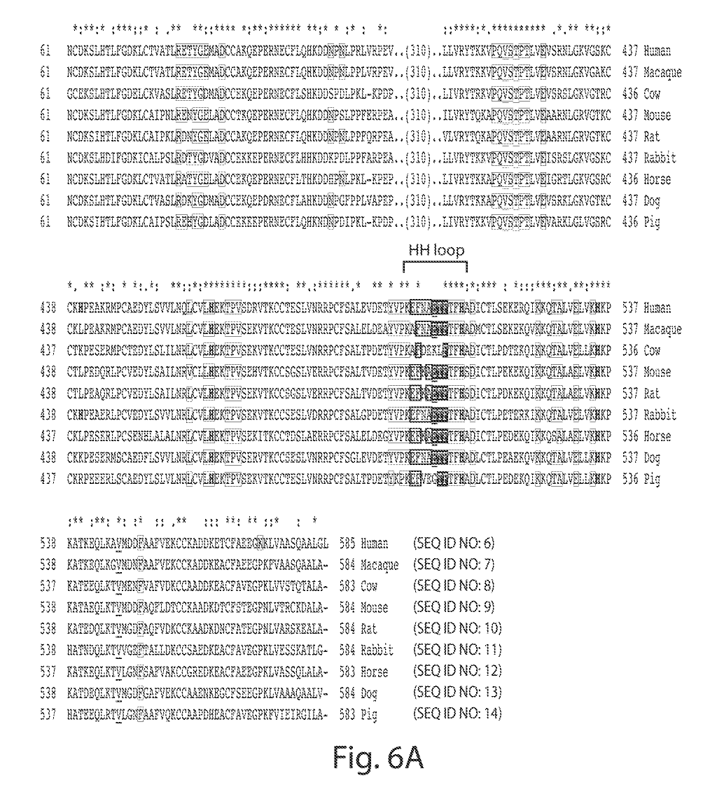

Albumin is a heart-shaped, wholly α-helical protein, while FcRn is closely related to MHC class I proteins, but with narrowed helices, such that peptides cannot be accommodated^{13,24,26} (FIGS. 5A and B). In the complex, the DIII and DI domains of HSA13 make spatially separated contacts to a single face of hFcRn, with DIII making a broad contact to the end of the α1α2 platform, the hinge, and β2m; and DI primarily contacting an exposed face of the α2 helix, in total burying 4068 Å² of surface area. Compared to ligands of other MHC class I-like proteins, the α1, hinge and β2m contact surface of hFcRn appears to be unique (Adams and Luoma (2013) Ann Rev Immunol 31:529-561).

A model of the HSA13/hFcRn/IgG ternary complex using the available rat FcRn/Fc structure (Martin et al. supra) shows at least 24 Å between any part of HSA and Fc, and even a rotationally free Fab arm should not be able to come any closer than 10 Å to HSA. This is consistent with the reported ability of FcRn to bind both ligands simultaneously with complete independence. It also indicates that a molecule can be designed that has altered binding properties to albumin but not to IgG.

The Ca backbone of hFcRn shows little movement compared to two other reported low pH structures (r.m.s.d. of 0.7 Å). On the other hand, HSA is a flexible protein, whose domains reportedly move to accommodate bound ligands. In HSA13, hFcRn binding induced several movements compared to apo HSA. DI and DIII each rotated compared to DII, and furthermore DIIIB rotated with respect to DIIIA, such that the DI-DIIIB distance across the cleft has increased by about 10 Å and the Arg-114/Glu-520 interdomain salt bridge cannot form. Individual DIIIB helices appear to move to maximize complementarity to hFcRn. Rotations in HSA domains upon hFcRn binding for HSA13 and apo HSA (PDB code 1AO6) were aligned on DII and are further illustrated in Schmidt et al. (supra) which is incorporated herein in its entirety, and further illustrates the side chains that can form an interdomain salt bridge in apo HSA. The mutation ^{HSA}K519E, away from the interface, reportedly eliminates hFcRn binding (Gao et al. U.S. Pat. No. 8,697,650) plausibly by stabilizing the DI-DIII contact via an ectopic salt bridge with Arg-186. Despite these movements, each domain is very similar to the corresponding domain in four other unliganded HSA structures (backbone Cα r.m.s.d. of 0.6-0.8 Å for DI, DII, DIIIA; 1.3-1.4 Å for DIIIB) A major displacement (≧2 Å) is seen in the flexible DIIIA-DIIIB connecting loop (the “DIII loop”).

The HSA13/hFcRn Interface

The HSA13/hFcRn interface is markedly hydrophobic (69% non-polar; Table 4) with polar contacts scattered throughout, consistent with the detergent sensitivity of the interaction. The DIII interface accounts for 76% of the contacts and 3076 Å² of buried surface area, while DI accounts for 24% and 1045 Å² respectively. This distribution is consistent with the reported unique ability of isolated HSA DIII to bind hFcRn, and reveals a role for DI in FcRn binding. Unexpectedly, two loops with high flexibility in crystal structures (the DIII loop, and the loop connecting the DIA carboxy-terminal helices²) are involved in FcRn contacts. These data indicate that in designing a variant HSA that is altered from the wild type in FcRn binding, both D1 and DIII features must be taken into account, for example, the two loops should be designed to retain flexibility if the variant is to retain FcRn binding.

TABLE 4
Interfacial contacts (≦4.5 Å cutoff distance). Polar contacts are in bold;
non-wild type amino acids are in italics; polar contacts to water are
bold and underlined.

| | HSA | | HSA | | HSA |
|---|---|---|---|---|---|
| hFcRnα | DI | hFcRnα | DIII | β2m | DIII |
| S58 | N109 | R42 | Y497, | R12 | F507, |
| | | | V498, | | [symbol] |
| | | | P499, | | |
| | | | K500 | | |
| W59 | N109 | E44 | P416, | S20 | K573 |
| | | | Q417, | | |
| | | | M418, | | |
| | | | Y497, | | |
| | | | K534 | | |
| K63 | N109 | E46 | K500 | N21 | K573 |
| N149 | E86, | P47 | K500 | F22 | A504, |
| | R81, | | | | G505, |
| | D89 | | | | K573 |
| K150 | E86 | G49 | T506, | E50 | E501, |
| | | | T508 | | F502, |
| | | | | | N503 |
| L152 | R81 | A50 | T508 | Y67 | N503, |
| | | | | | T506 |
| T153 | G85, | V52 | T527, | E69 | K573 |
| | E86, | | E531 | | |
| | R81 | | | | |
| L156 | R81, | W53 | F507, | | |
| | E82 | | T508, | | |
| | | | F509, | | |
| | | | K524, | | |
| | | | T527, | | |
| | | | A528, | | |
| | | | F551 | | |
| F157 | R81, | E54 | K524 | | |
| | E82, | | | | |
| | T83, | | | | |
| | Y84, | | | | |
| | G85 | | | | |
| H161 | E82 | N55 | K524 | | |
| E165 | N111 | Q56 | P421 | | |
| | | V57 | S419, | | |
| | | | P421 | | |
| | | S58 | P421, | | |
| | | | T422, | | |
| | | | E425 | | |
| | | W59 | M418, | | |
| | | | T422, | | |
| | | | E425, | | |
| | | | L460, | | |
| | | | L463, | | |
| | | | H464, | | |
| | | | T467 | | |
| | | W61 | S419 | | |
| | | E62 | M418, | | |
| | | | S419, | | |
| | | | T422, | | |
| | | | T467, | | |
| | | | V469 | | |
| | | K63 | T467 | | |
| | | T66 | T467, | | |
| | | | P468 | | |
| | | R69 | P468, | | |
| | | | V469 | | |
| | | G172 | D512 | | |
| | | N173 | H510 | | |
| | | W176 | H510 | | |
| | | S230 | [symbol] | | |
| | | | T506 | | |
| | | D231 | [symbol] | | |
| | | | T508 | | |

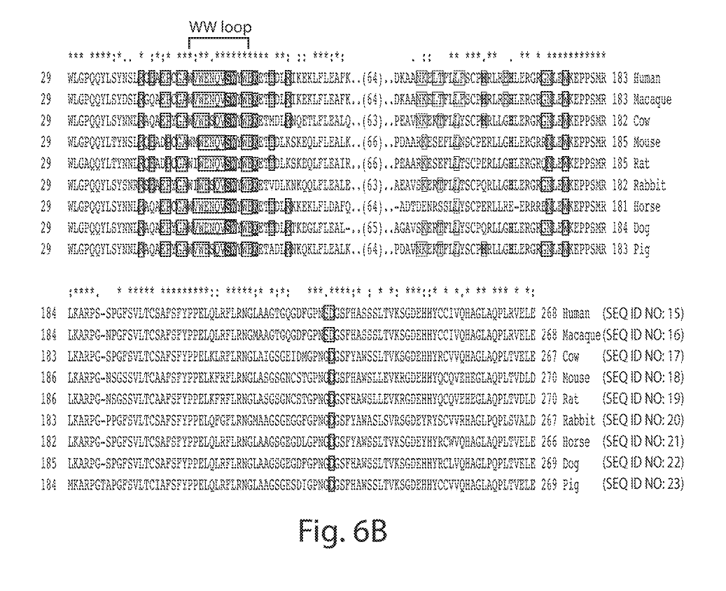

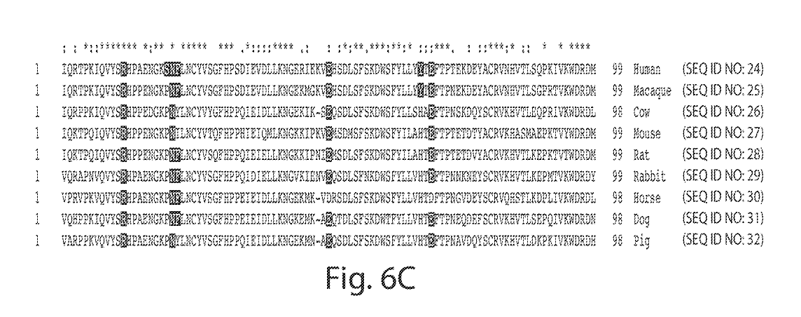

In the DIII contact, the most striking feature is the insertion of two absolutely conserved FcRn tryptophans, Trp-53 and Trp-59 (FIG. 6B), into deep hydrophobic pockets in DIII (“W53-” and “W59 pockets”), burying about 180 and 160 Å² of surface area, respectively (FIG. 2A). Trp-53 and -59 are located in a loop in the FcRn α1 domain (from Trp-51 to Trp-61) that is termed herein the “WW loop”.

The DIII contact can be further subdivided into the DIIIA and the DIII loop/DIIIB contacts. DIIIA primarily contains the W59 pocket, but also makes a number of stabilizing contacts to the surface of the FcRn α1 helix (Table 4). The W59 pocket lies at the aliphatic end of the fatty acid binding site 4 (FA4) in DIIIA³⁰, and, compared to unbound low pH structures, the ^{hFcRnα}W59 side chain has to flip and rotate about 100° to make this interaction (FIG. 3A). The W59 pocket is wider than both defatted and fatted structures (by about 2 Å and 1 Å, respectively). This could be due to insertion or due to an effect of two changes in HSA13 that occur near the W59 pocket (V418M, T420A). Met-418 extends the hydrophobic surface against which Trp-59 packs, while the reduced side chain of Ala-420, which increases affinity significantly by itself (Table 2), allows DIIIA helix h2 to move towards DIIIB helix h2. A V424I mutation, one turn further down than T420A in the DIIIA-h3 helix, also increases affinity, presumably through better packing against helix DIIIB-h2 (Table 2).

To confirm the role of Trp-59 in HSA interaction, we replaced the side chain with another aromatic residue (Phe) or a methyl group (Ala). Strikingly, ^{hFcRnα}W59A abolishes both HSA13 and HSA binding, while the affinity of ^{hFcRnα}W59F for both is reduced, but not eliminated (FIG. 3B).

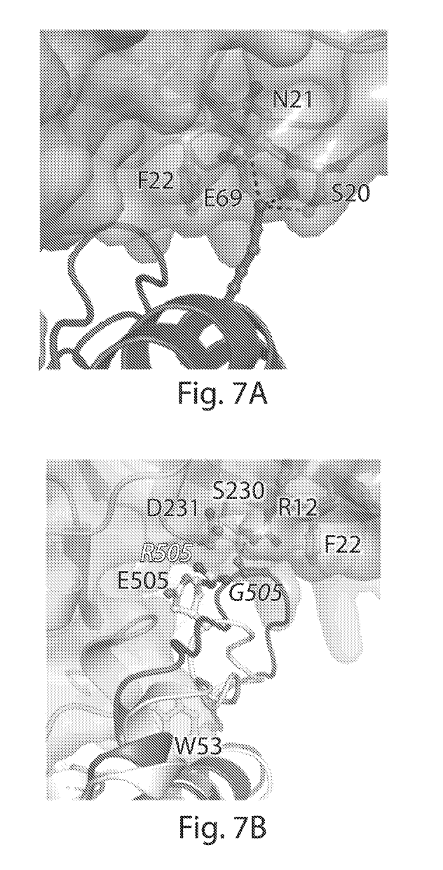

The DIII loop/DIIIB contact contains the W53 pocket, and its formation requires a unique displacement of part of the DIII loop (residues Lys-500 to His-510; the “HH loop”) induced by a steric clash with the WW loop of hFcRn (FIGS. 2A and 4B). The W53 pocket lies very close to thyroxine binding site Tr3³¹, and the W53 side chain has a similar disposition to one ring of thyroxine (FIG. 3d), having rotated 10-25° compared to unbound hFcRn. In this region additional HSA13 contacts are made to the WW loop, the α1 platform, α3 and β2m (Table 4). Notably, a single side chain in the HSA13 terminal helix (Lys-573) makes one of the two interfacial salt bridges to ^{β2m}Glu-69 (FIG. 7A). ^{HSA}Lys-573 is apparently unique to humans (being proline in almost every other species; FIG. 6A), and is a reportedly sensitive site for hFcRn affinity.

The HH loop contains one of the four HSA13 changes (E505G), which in isolation produces a three-fold affinity improvement (Table 2). Backbone atoms of Gly-505 make favorable polar contacts to ^{hFcRnα}Ser-230 in the α3 domain and ^{β2m}Arg-12, as well as a non-polar contact to ^{β2m}Phe-22. In wild type HSA, the large negative side chain of Glu-505 would reduce complementarity but likely restore this part of the HH loop to better resemble apo HSA (FIG. 7B). The E505R mutation has a positive effect similar to E505G, potentially due to Arg-505 interacting with ^{hFcRnα}D231 (see FIG. 7B). I523G, which increases affinity by about 40-fold²⁸, lies in the helix DIIIB-h2 that forms part of the W53 pocket. Without committing to any particular theory, applicants attribute this positive effect to arise from an introduced kink in that helix, right at the W53 pocket, improving W53 fit.

As with Trp-59, a W53F mutation is well tolerated with only a modest change in affinity, while the loss of the side chain in a W53A mutant completely abolishes hFcRn binding to both HSA13 and HSA (FIG. 3E). Upon insertion, Trp-53 makes a transverse π-stacking interaction with Phe-509 (FIG. 3D). Mutational analysis supports the importance of this contact in complex stability. An F509M mutation in HSA, which would lose the stacking interaction and alter the packing in the pocket, has a 30-fold reduction in FcRn binding, while the tryptophan side chain in a F509W mutation would directly compete with Trp-53, and abolishes hFcRn binding (Gao, U.S. Pat. No. 8,697,650).

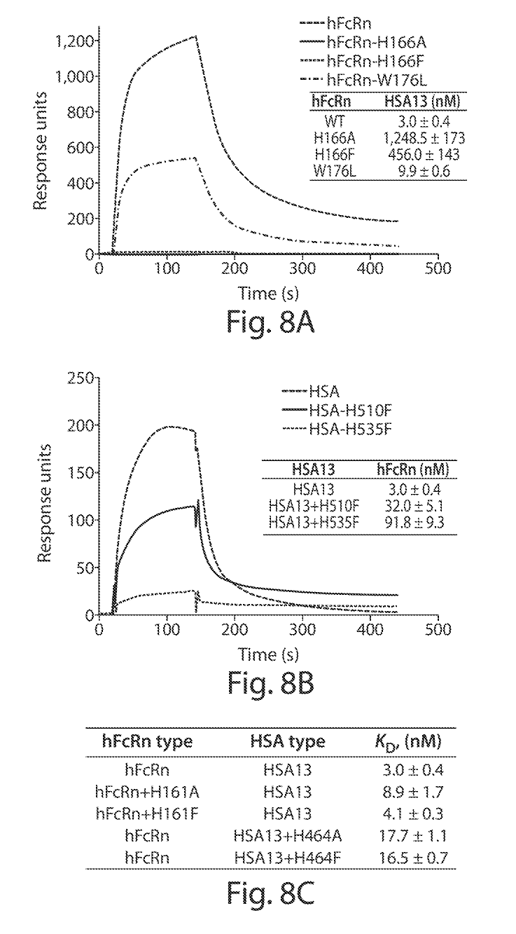

The DI contact is almost exclusively confined to the loop between DIA-h4 and h5, and the α2 helix of FcRn (FIG. 2D). His-161, which has been suggested to form part of the pH sensor in hFcRn¹⁸, could make a hydrogen bond to the backbone carbonyl of ^{hFcRnα}E82. However, mutation of His-161 to Ala has little effect on HSA binding¹⁸ (FIG. 8); and H161 is poorly conserved (FIG. 6B), all of which fail to support a role in pH sensing.

Accordingly, the ability of an HSA variant to interact with Trp residues, e.g., Trp-53 and Trp-59 of FcRn is important to preserving and/or increasing affinity of the HSA variant to FcRn.

Mechanism of pH-Dependent Binding

The WW loop of hFcRn makes no HSA contacts that would vary across the pH 6.0-7.4 range, but the loop itself is stabilized in hFcRn by a protonated ^{hFcRnα}His-166, anchoring a network of hydrogen bonds (FIG. 4A). His-166 is absolutely conserved in FcRn (FIG. 6B) but makes no direct HSA contact. Protonation of His-166 has been previously identified as a candidate part of the pH sensor, and its effect on the WW loop has been noted^{10,17,18}. Either ^{hFcRnα}H166F or ^{hFcRnα}H166A abolish wild type HSA binding, and reduce HSA13 affinity >100-fold (FIG. 8).

The HH loop is anchored at each end by a series of hydrogen bonds and π-cation interactions involving protonated HSA histidines at 510 and 535 (FIG. 4b-d), both of which are absolutely conserved (FIG. 6A). ^{HSA}H510 forms the sole intermolecular, potentially pH-sensitive interaction as a π-cation contact to the absolutely conserved ^{hFcRnα}W176 residue (FIG. 4C and FIG. 6B), and mutation of ^{hFcRnα}W176 to leucine produces a 3-fold reduction in binding affinity (FIG. 8A). All other His-510 and His-535 interactions are intramolecular and likely act to stabilize the HH loop. Consistent with this, mutation of His-510 or -535 in HSA13 to Phe results in a 10- and 30-fold loss of affinity for hFcRn, respectively while in wild type HSA, H353F nearly eliminates hFcRn binding (FIG. 8).

NMR studies have shown that in DIII at pH 6, only two of the four histidines will be protonated (Bos et al. J Biol Chem 264:953-959; Labro and Janssen (1986) Biochim Biophys Acta 873:267-278), which applicants believe to be His-510 and -535. Mutational studies have shown that changes at His-464 also reduce hFcRn affinity (Andersen et al. (2012) Nature Communications 3:610) (FIG. 8). However, His-464 contacts Trp-59 in the W59 pocket, and is buried in a hydrophobic environment. His-464 shows no evidence of inter- or intramolecular ionic side-chain interactions or movement in HSA13 or 66 other HSA structures, and likely remains unprotonated.

Applicants have therefore constructed a model for the pH-dependent interaction of hFcRn and HSA in which at pH 7.4, the WW loop is unstructured, and the HH loop is loosely structured. Upon shift to pH 6.0, ^{hFcRnα}His-166 protonates and stabilizes the WW loop, holding Trp-53 and Trp-59 at the hFcRn surface. At the same time, ^{HSA}His-510 and ^{HSA}His-535 become fully protonated and anchor the HH loop in a more “open” position. Interaction is stabilized by ^{hFcRnα}W59 rotation into the W59 pocket, the DI contact and ^{HSA}His-510 binding to ^{hFcRnα}Trp-176. Finally, the hFcRnα3 and β2m interaction with the DIIIB/DIII loop contact rotates it “up” fully opening the W53 pocket, allowing ^{hFcRnα}Trp-53 insertion (FIG. 4E).

In this model, the main barrier to neutral pH binding is the cost of stabilizing the WW loop and opening the HH loop. It is therefore possible to predict that changes that increase binding at pH 6.0 by strengthening existing contacts also increase affinity at pH 7.4. This is supported by the finding that four variants quantitated by SPR had micromolar hFcRn affinity at pH 7.4 (Table 1). Table 1 shows kinetic data for selected HSA variants generated by applicants binding to immobilized single-chain hFcRn at pH 6.0 and 7.4 as measured by surface plasmon resonance (NM, not meaningful; data are from curves fitted by the supplied software). Fc mutations that increase the affinity of hIgG1 for hFcRn typically have a proportionate effect on binding across all pHs²¹. Surprisingly, the HSA variants reported here show that the relationship between pH 6.0 and pH 7.4 binding can be uncoupled for HSA (compare HSA11 and 7; Table 1).

Further, data provided herein demonstrate that intramolecular histidine contacts drive the pH switch, e.g., protonated His-166 on hFcRn stabilizes the “WW-loop” and protonated His-510 and His-535 on HSA stabilize the “HH-loop” and open the W53 pocket.

### Example 5

### hFcRn Competition with Ligands

Based on a superposition of 35 structures with fatty acid present in FA4, Trp-59 would be unable to rotate into its pocket in the presence of C16 or C18 lipids (FIG. 3A). Similarly, based on a superposition of three structures with thyroxine (T4) present in Tr3, Trp-53 would be unable to insert into the W53 pocket in the presence of thyroxine (FIG. 3D), indicating that both key FcRn interactions may be compromised in the presence of certain SA ligands, e.g., long chain fatty acids. To further test this, the ability of FA to compete with hFcRn binding to HSA at pH 6.0 was examined. Strikingly, C16:0 and C18:1 FAs abolished hFcRn binding, while C12:0 had a lesser effect (FIG. 3C and FIG. 3F), revealing that the recycling efficiency of ligand-bound HSA is likely to be lower than ligand-free HSA. Due to the low solubility of T4 and its low affinity for HSA, the corresponding effect at the W53 pocket was not demonstrated.

These data demonstrate that one means of delivering ligands to cells is via non-salvage followed by lysosomal degradation of albumin.

Pharmacokinetic Analysis of HSA Variants

For IgGs, increasing FcRn affinity can increase the circulating half-life, although the relationship is not strict. Applicants examined the half-lives of some of the HSA variants in mice and primates (Table 5) using hemagglutinin-tagged HSA and HSA variants at doses of 5 mg/ml and 1 mg/ml. HSA species were directly quantitated by ELISA. In wild type C57B/J mice, HSAs 11 and 13 showed increases of 41 and 53% in the elimination half life, t_{1/2}, compared to HSA, respectively, while HSA5 and 7 were more similar to HSA.

TABLE 5
Pharmacokinetic parameters.

| | | | | | | | |
|---|---|---|---|---|---|---|---|
| | Dose | | Cₘₐₓ | AUC | C1 | t_{1/2β} | P value |
| Molecule | (mg/kg) | N | (μg/ml) | (μg · h/ml) | (ml/min/kg) | (h) | for t_{1/2β} |

a)

| | | | | | | | |
|---|---|---|---|---|---|---|---|
| HSA | 2.5 | 3 | 34.2 | 582 | 0.080 | 30.3 | — |
| | | | (26.7-44.5) | (496-633) | (0.073-0.093) | (29.2-31.4) | |
| HSA5 | 2.5 | 3 | 32.3 | 825 | 0.058 | 46.2 | 0.172 |
| | | | (17.9-43.9) | (641-937) | (0.049-0.072) | (37.4-61.3) | |
| HSA7 | 2.5 | 3 | 22.6 | 664 | 0.071 | 44.9 | 0.020 |
| | | | (16.4-26.8) | (564-791) | (0.059-0.082) | (42.0-49.6) | |
| HSA11 | 2.5 | 3 | 25.0 | 564 | 0.083 | NM | — |
| | | | (22.8-27.1) | (497-629) | (0.074-0.093) | | |
| HSA13 | 2.5 | 3 | 22.9 | 249 | 0.187 | NM | — |
| | | | (20.1-27.9) | (230-272) | (0.170-0.201) | | |

b)

| | | | | | | | |
|---|---|---|---|---|---|---|---|
| HSA | 2.5 | 3 | 34.2 | 582 | 0.080 | 30.3 | — |
| | | | (26.7-44.5) | (496-633) | (0.073-0.093) | (29.2-31.4) | |
| HSA5 | 2.5 | 3 | 32.3 | 825 | 0.058 | 46.2 | 0.172 |
| | | | (17.9-43.9) | (641-937) | (0.049-0.072) | (37.4-61.3) | |
| HSA7 | 2.5 | 3 | 22.6 | 664 | 0.071 | 44.9 | 0.020 |
| | | | (16.4-26.8) | (564-791) | (0.059-0.082) | (42.0-49.6) | |
| HSA11 | 2.5 | 3 | 25.0 | 564 | 0.083 | NM | — |
| | | | (22.8-27.1) | (497-629) | (0.074-0.093) | | |
| HSA13 | 2.5 | 3 | 22.9 | 249 | 0.187 | NM | — |
| | | | (20.1-27.9) | (230-272) | (0.170-0.201) | | |

c)

| | Dose | | Cₘₐₓ | AUC | C1 | | Mean | Mean | P value |
|---|---|---|---|---|---|---|---|---|---|
| Molecule | (mg/kg) | N | (μg/ml) | (μg · h/ml) | (ml/min/kg) | t_{1/2β}(h) | C1 | t_{1/2β} | for t_{1/2β} |
| HA-HSA | 1 | 6 | 18.2 | 1,810 | 0.0100 | 153 | 0.0090 | 169 | — |
| | | | (11.3-27.1) | (1,220-2,710) | (0.0061-0.0138) | (119-226) | | | |
| HA-HSA | 5 | 2 | 238 | 15,300 | 0.0059 | 218 | | | |
| | | | (182-294) | (10,900-19,700) | (0.0072-0.0076) | (180-257) | | | |
| HA-HSA7 | 1 | 2 | 38.1 | 2,820 | 0.0059 | 239 | 0.0053 | 259 | 0.003 |
| | | | (36.2-40.0) | (2,750-2,900) | (0.0057-0.0061) | (225-254) | | | |
| HA-HSA7 | 5 | 2 | 196 | 17,900 | 0.0047 | 279 | | | |
| | | | (187-205) | (16,100-19,700) | (0.0042-0.0052) | (265-294) | | | |

a) Wild type C57/B6 mice.
b) C57BL/6-Derived mFcRn-/- mice homozygous for an hFcRn transgene.
c) Cynomolgus macaques.
Values are means (ranges) of curves fitted for each animal.
P values (t-distribution; 2-tailed, unequal variance) are for elimination half-life differences between HSA variants and wild type HSA.
NM, not meaningful.

On the other hand, in mFcRn^{-/-} mice transgenic for hFcRn (Petkova et al. (2006) International Immunol 18:1759-1769), HSA5 and HSA7 showed 52% and 48% increases respectively in t_{1/2}, while HSA11 and 13 had reduced t_{1/2} and elevated clearance due to an antibody response, presumably arising from their pH 7.4 hFcRn affinity. In macaques, HSA7 showed an increase of 53% in t_{1/2} and a 41% reduction in clearance compared to wild type HSA. Thus, affinity for hFcRn at pH 6.0 is a primary determinant of circulating half-life and a component of design for an HSA variant having increased half-life.

### Example 6

### Susceptibility of DIII Histidines to Protonation

There are four histidines in HSA DIII (at positions 440, 464, 510 and 535) whose titration has been examined using NMR (Bos et al. supra; Labro and Janssen supra). Four C-2 proton resonances have been assigned to the DIII histidines, and they show unique behavior. Resonance 6 titrates readily, with a pK*ₐ above 7.5, resonance 9 titrates with difficulty, at a pK*ₐ of ˜5, resonance 11 has a broad signal that titrates at a more typical pK*ₐ of 6-6.5, and resonance 12 does not titrate at all, even at pH 5.0, and is interpreted to be shielded from solvent.

Of the four histidines, His-464 is the only one that appears buried, and its position and contacts do not change between the apo form, the pH 4.9 hFcRn-bound form, any ligand-bound form, or in another pH 6 dilapidated structure (PDB code 1TF0). His-464 contacts W59; furthermore, protonation would be expected to reduce the hydrophobic environment of the pocket. We therefore believe His-464 corresponds to resonance 12. His-440 is on the surface but located in a cluster of basic residues (K436/K439/K444/R445), which should decrease the local pK*ₐ. His-440 is also not conserved (FIG. 6A), making unlikely to be part of a pH sensing mechanism. Applicants therefore assign resonance 9 to His-440. Clipping of a tryptic fragment of HSA near residue 413/414 affects the ionization of this resonance, as does diazepam binding (Bos et al. supra). Both of these sites lie closest to His-440¹, supporting the assignment of this resonance. His-535 is near the surface of HSA and adopts one of two conformations in available structures. In seven structures solved at pH 7.0-7.5 (PDB codes 1E7A, 1E7F, 2XW1, 2BXG, 1AO6, 1BM0, 2VUE), His-535 appears to be making hydrogen bonds to the backbone carbonyls of either Lys-500 and Lys-534, or Glu-501 and Glu-531 (as it does in our pH 4.9 structure and the pH 6 1TF0 structure), consistent with His-535 being protonated and corresponding to resonance 6. Finally, His-510 is fully exposed to solvent, and likely has the most normal pK*ₐ, suggesting that His-510 corresponds to the broad resonance 11.

Structural information provided herein can be used in methods to make HSA variants that themselves have increased circulating half-life and/or confer an increase in circulating half-life on molecules bound, e.g., fused to such an HSA variant.

The skilled artisan, having read the above disclosure, will recognize that numerous modifications, alterations of the above, and additional optimization of the above, may be conducted while remaining within the scope of the invention. These include but are not limited to the embodiments that are within the scope of the following claims.

1. A method of identifying a human serum albumin (HSA) variant, the method comprising
a. providing a mutated HSA; and
b. determining whether the mutated HSA has at least one mutation in domain III that decreases fatty acid binding compared to fatty acid binding by a wild type HSA, wherein a mutated HSA that decreases fatty acid binding compared to a wild type HSA is an HSA variant.

2. The method of claim 1, further comprising
c. determining the binding affinity of the mutated HSA for FcRn,
wherein a mutated HSA that can bind to FcRn with the same or increased affinity compared to binding of a wild type HSA to FcRn is an HSA variant.

3. The method of claim 1, further comprising
d. determining the PK of the mutated HSA compared to the PK of a wild type HSA,
wherein a mutated HSA that has increased PK compared to a wild type HSA is an HSA variant.

4. A human serum albumin (HSA) variant comprising at least one mutation in domain III that decreases fatty acid binding to the HSA variant compared to fatty acid binding by a wild type HSA.

5. The HSA variant of claim 4, wherein the HSA variant can bind to FcRn.

6. The HSA variant of claim 4, wherein the HSA variant has an increased PK compared to a wild type HSA.

7. The HSA variant of claim 4, wherein the mutation
a. alters one or more residues in domain III of a wild type HSA that can bind to a carboxyl; or
b. alters one or more residues in domain III that are lining residues.

8. The HSA variant of claim 7, wherein the HSA variant is mutated at one or more residues selected from the group consisting of R410, Y411, S489, Y401, and K525.

9. The HSA variant of claim 8, wherein the residue is mutated to a non-polar amino acid or a negatively charged amino acid.

10. The HSA variant of claim 9, wherein the mutated residue is an alanine or glutamic acid.

11. The HSA variant of claim 7, wherein the mutated residue is a lining residue selected from the group consisting of Y411, V415, V418, T422, L423, V426, L430, L453, L457, L460, V473, R485, F488, L491, F502, F507, F509, K525, A528, L529, L532, V547, M548, F551, L575, V576, S579, and L583.

12. The HSA variant of claim 11, wherein the mutated residue is selected from the group consisting of Y411, V415, V418, L423, V426, L430, L453, L457, L460, V473, P485, F488, L491, F502, F507, F509, A528, L529, L532, V547, M548, F551, L575, V576, and L583.

13. The HSA variant of claim 12, wherein the mutated residue is mutated to a serine.

14. An HSA variant covalently linked to a therapeutic agent.

15. A method of identifying a scaffold molecule, the method comprising
a. providing a candidate molecule; and
b. determining whether the candidate molecule can bind to an HSA and can inhibit fatty acid binding to the HSA,
wherein, a candidate molecule that can bind to an HSA and can inhibit fatty acid binding is a scaffold molecule.

16. The method of claim 15, wherein the scaffold molecule can bind to one or more of residues R410, Y411, S489, Y401, or K525 of a wild type HSA.

17. A scaffold molecule identified by the method of claim 15.

18. The scaffold molecule identified by the method of claim 17 further comprising a therapeutic molecule, thereby forming a heterogeneous scaffold molecule, wherein the PK of the heterogeneous scaffold molecule is increased compared to the PK of the therapeutic molecule.

19. A method of increasing the serum half-life of a molecule, the method comprising covalently linking the molecule to the HSA variant of claim 4.

20. The method of claim 19, wherein the molecule is a protein or polypeptide.

21. A molecule comprising the HSA variant of claim 4 and a heterologous molecule.

22. The molecule of claim 21, wherein the heterologous molecule is a protein or polypeptide.
